(19) United States
(12) Patent Application Publication
(10) Pub. No.: US 2025/0090706 A1
(43) Pub. Date: Mar. 20, 2025

(54) DISINFECTION ROBOT, SYSTEM FOR DISINFECTION, AND METHOD OF DISINFECTION

(71) Applicant: RYBERG IP B.V., Amsterdam (NL)

(72) Inventor: Man Yong TOH, Amsterdam (NL)

(21) Appl. No.: 18/290,826

(22) PCT Filed: Jul. 23, 2022
(86) PCT No.: PCT/NL2022/050436

§ 371 Date: Jan. 22, 2024

(30) Foreign Application Priority Data
Jul. 23, 2021 (NL) 2028835

(51) Int. Cl. A61L 2/24; A61L 2/10; A61L 2/26; A61L 9/20; G05D 1/648; G05D 1/689; G05D 107/60

(52) U.S. Cl. CPC A61L 2/24; A61L 2/10; A61L 2/26; A61L 9/20; G05D 1/648; G05D 1/689; A61L 2202/11; A61L 2202/14; A61L 2202/16; A61L 2202/25; A61L 2209/111; A61L 2209/12; A61L 2209/15; G05D 2107/65

(57) ABSTRACT

Disinfection robot for disinfecting a space by means of ultraviolet radiation, which includes a radiation source for emitting the ultraviolet radiation, movement means for moving the radiation source in the space, and sensor means for generating a sensor signal that is indicative for a position of the radiation source in the space. The robot includes a controller that is arranged for receiving the sensor signal and that is arranged for controlling the movement means for moving the radiation source along a path in the space. The controller is arranged for determining a cumulative exposure pattern of the emitted ultraviolet radiation in the space. The controller is arranged for determining a target position to extend the path based on the sensor signal and on the cumulative exposure pattern in the space. and for controlling the movement means for moving the radiation source towards the target position.

### FIELD

The invention relates to a disinfection robot for disinfecting a, preferably indoor, space such as a room of a hospital or a nursing centre, by means of ultraviolet radiation. The invention also relates to a system that includes a disinfection robot and a remote computer. The invention further relates to a method of disinfecting a, preferably indoor, space such as a room of a hospital or a nursing centre, by means of ultraviolet radiation. The invention also relates to a computer program product, to a computer-readable storage medium, and to use of a disinfection robot.

### BACKGROUND

The importance of risks associated with pathogens and allergens are nowadays recognised more and more. This is not only due to virus outbreaks on a large scale, but also because of the general level of medical care that has increased during the last decades. In addition, new types or variations of pathogens and allergens can occur and spread, so that a continuous need for effective disinfection is nowadays generally recognised.

Disinfection efforts may be directed to various kinds of spaces where people reside, such as trains, planes, restaurants etc. Such spaces may benefit from disinfection because of the large number of different people that may be present there, which generally exposes people at such places to a relatively large risk. A particularly high benefit of disinfection may be present in medical spaces such as hospitals and nursing centres. After all, hospital rooms and nursing centres are visited by many different people that moreover may be relatively vulnerable.

At the same time, medical spaces such as hospital rooms or nursing centres are relatively expensive to maintain, because of the specialised equipment that can be present in such spaces and the specialised personnel working in such spaces. For example, idle hospital rooms may generally be considered costly. Thus, while infection of hospital rooms and other medical spaces requires relatively high standards of disinfection, the period of availability of such spaces is important as well. After all, during disinfection, a hospital room or other medical space may not be available for providing medical care.

Traditionally, medical spaces are disinfected manually without the help of a robot. However, manual disinfection is not always reliable, due to the possibility of human error. Moreover, a human performing disinfection may be at risk from pathogens and/or allergens in the room, which is undesirable. In addition, humans may spread a pathogen and/or allergen to another space. Also, manual disinfection can be considered to be relatively expensive and slow, when it is necessary to achieve a high standard of disinfection. After all, a thorough manual disinfection of a room can be expected to take a relatively large amount of time.

International patent application WO 2015/116876 describes a device for disinfecting a hospital room. The device includes a lamp assembly for emitting ultraviolet light, a mobile base to manoeuvre the device and a distance measuring sensor. The device is provided with a microprocessor that determines disinfection parameters such as disinfection time, using a look-up table that can be established on the basis of computer simulations of ultraviolet light in the hospital room. WO 2015/116876 does not disclose improving the control of the movement of the device in the hospital room.

There is a need for an improved disinfection device and method of disinfection, that can disinfect a space of, for example, a hospital or a nursing centre, in a reliable way and in an acceptable time. At least, there is a need for an alternative disinfection device and an alternative method of disinfection.

### SUMMARY

The invention provides a disinfection robot for disinfecting a, preferably indoor, space such as a room of a hospital or a nursing centre, by means of ultraviolet radiation, wherein the disinfection robot is arranged to include, and preferably includes, a radiation source that is arranged for emitting the ultraviolet radiation, wherein the disinfection robot also includes movement means for moving the radiation source in the space and sensor means for generating a sensor signal that is indicative for a position of the radiation source in the space, wherein the robot further includes a controller that preferably is arranged for receiving the sensor signal and/or preferably is communicatively connected to the sensor means for receiving the sensor signal, and that is arranged for controlling the movement means for moving the radiation source along a path in the space. Said path may be formed by a sequence of the positions of the radiation source in the space. Preferably, the controller is arranged for receiving the sensor signal via a communicative connection between the sensor means and the controller.

According to an aspect, the controller is further arranged for determining a cumulative exposure pattern of the emitted ultraviolet radiation in the space, preferably based on the path, on a duration and/or velocity of movement of the radiation source along the path, and/or on an intensity and/or direction of the ultraviolet radiation emitted during movement along the path. According to another aspect, the controller is arranged for determining a target position to extend the path in the space based on the sensor signal and on the cumulative exposure pattern in the space, and for controlling the movement means for moving the radiation source towards the target position.

Determining the target position based on the sensor signal and the cumulative exposure pattern, enables the controller to set a target position for the radiation source at a position where an improvement of the cumulative exposure pattern can be reached at a relatively short distance, and hence in a relatively short time. Instead of traversing the room along a path that is fully determined by a human, determining the target position based on the sensor signal and the cumulative exposure pattern thus enables a robust manner to disinfect the space that takes into account the duration of disinfecting and the level of disinfecting. After all, the cumulative exposure pattern indicates which portions of the space were exposed to the ultraviolet radiation and indicates the total amount of ultraviolet radiation that was received as a result of the disinfecting.

Preferably, the cumulative exposure pattern associates a position in the space with a received amount of ultraviolet radiation as a result of the disinfecting. Preferably, determining the exposure pattern based on the intensity of the ultraviolet radiation takes into account a decrease of the intensity with increasing distance from the radiation source. Preferably, determining the exposure pattern based on the intensity of the ultraviolet radiation takes into account a decreasing radiant power of the radiation source with time. Such variations in intensity are preferably determined experimentally and/or by modelling, before using the radiation source for the disinfecting. The controller preferably is arranged for repeatedly determining the cumulative exposure pattern, during disinfecting the room. Preferably, the cumulative exposure pattern is updated before determining the target position, preferably is updated repeatedly or each time before determining the target position. Preferably, the controller is arranged for storing a sequence of the cumulative exposure patterns.

In an embodiment, the controller is arranged for determining a position of the radiation source in the space, based on the sensor signal that is indicative for a position of the radiation source in the space. Optionally, such determining is based on an indication or estimate of the radiation source position. Preferably, the controller is arranged for determining the target position to extend the path in the space based on the position of the radiation source and on the cumulative exposure pattern.

In an embodiment, the controller is arranged for determining the target position based on a distance of the target position from the radiation source position and on a received amount of ultraviolet radiation at the target position. Thus, in an embodiment, the target position is based on a combination of its distance from the radiation source position and its received amount of ultraviolet radiation. Based on the sensor signal that is indicative for a position of the radiation source in the space, the controller preferably determines the position of the radiation source. The position of the radiation source enables determining the distance from the radiation source to the target position.

In an embodiment, the cumulative exposure pattern includes a plurality of space portions that are associated with a position of the space portion in the space and with an amount of ultraviolet radiation received in the space portion as a result of the disinfecting. Optionally, such association may be established by means of a data structure in a memory of the controller, wherein the plurality of space portions are individually linked to a position in the space and to the received amount of ultraviolet radiation in the space portion. The space portions may for example be defined by rectangular elements of a two-dimensional grid or box-shaped elements of a three-dimensional grid that extends in the space. Preferably, the controller is arranged for determining the target position in a target space portion of the plurality of space portions, based on a combination, in the target space portion, of distance from the radiation source position and received amount of ultraviolet radiation.

In an embodiment, the controller is arranged for determining the target position in a target space portion of the plurality of space portions that is associated with a lower combination of distance from the radiation source position and received amount of ultraviolet radiation, than another space portion in the space, preferably lower than another space portion positioned in a, preferably predetermined, range from said target space portion, more preferably lower than another space portion positioned adjacent to the target space portion. Thus, preferably, for the target space portion the combination of its distance from the radiation source position and its received amount of ultraviolet radiation, is lower than in another space portion, preferably lower than another space space portion in a, preferably predetermined, range from the target space portion, more preferably lower than a space portion adjacent to the target space portion.

It may thus be clear that, in an embodiment, the controller is arranged for determining the target position in a target space portion of the plurality of space portions that is associated with a lower combination of distance from the radiation source position and received amount of ultraviolet radiation, than another space portion in the space. Hence, in an embodiment, for at least one space portion in the space said combination is higher than for the target space portion. In a further embodiment, the controller is arranged for determining the target position in a target space portion of the plurality of space portions that is associated with a lower combination of distance from the radiation source position and received amount of ultraviolet radiation, than another space portion positioned in a, preferably predetermined, range from said target space portion. Hence, in a further embodiment, for at least one space portion in said range said combination is higher than for the target space portion. In a yet further embodiment, the controller is arranged for determining the target position in a target space portion of the plurality of space portions that is associated with a lower combination of distance from the radiation source position and received amount of ultraviolet radiation, than another space portion positioned adjacent to the target space portion. Hence, in a yet further embodiment, for at least one space portion adjacent to the target space portion said combination is higher than for the target space portion.

Preferably, the range from the target space portion is defined by a geometric shape, such as a circle or square. Said shape may be approximately at the position of the disinfection robot and/or of the radiation source. A maximum cross section length, such as a maximum diameter, of said geometric shape, may for example be at least 1 meter and/or at most 10 meter. As another example, the cross section length may be at least 1.5 times, or at least two times, and/or at most ten times, a maximum horizontal cross section length, such as a diameter, of a housing of the robot or of the target space element.

Said combination of distance from the radiation source position and received amount of ultraviolet radiation, preferably is proportional, more preferably substantially linearly proportional, to the distance from the radiation source and to the cumulative amount of received ultraviolet radiation. Experiments show that selecting a minimum, relative to one or more other space portions, for said combination in the target position, enables an efficient way of disinfecting the space.

For example, the target space portion may be relatively close to the radiation source position so that an improvement in the cumulative exposure pattern can be reached relatively quickly. As another example, the target space portion may have received a relatively low amount of ultraviolet radiation, so that a significant improvement in the cumulative exposure pattern may be reached.

In an embodiment, the controller is arranged for determining the target position in a target space portion of the plurality of space portions that is associated with a lower combination of distance from the radiation source position and received amount of ultraviolet radiation, than a majority, preferably all, of the space portions that are positioned adjacent to the target space portion, preferably lower than a majority, preferably all, of the space portions positioned in a, preferably predetermined, range from the target space portion, more preferably lower than a majority, preferably all, of the plurality of space portions positioned in the space. Thus, in an embodiment, in the space, in the range from the target space portion, and/or adjacent to the target space portion, the target space portion may be associated with a minimum of the combination of distance from the radiation source position and received amount of ultraviolet radiation. Taking into account a larger number of the plurality of space portions for selecting the target space portion, may enable selecting a lower minimum of said combination associated with the target position. By taking into account more of the space portions, a chance that said combination is lower in another space portion than in the target space portion, may decrease.

It may thus be clear that, in an embodiment, the controller is arranged for determining the target position in a target space portion of the plurality of space portions that is associated with a lower combination of distance from the radiation source position and received amount of ultraviolet radiation, than a majority, preferably all, of the space portions that are positioned adjacent to the target space portion. Hence, in an embodiment, for a majority, preferably all, space portions adjacent to the target space portion said combination is higher than for the target space portion. In a further embodiment, the controller is arranged for determining the target position in a target space portion of the plurality of space portions that is associated with a lower combination of distance from the radiation source position and received amount of ultraviolet radiation, than a majority, preferably all, of the space portions positioned in a, preferably predetermined, range from the target space portion. Hence, in a further embodiment, for a majority, preferably all, space portions in said range said combination is higher than for the target space portion. In a yet further embodiment, the controller is arranged for determining the target position in a target space portion of the plurality of space portions that is associated with a lower combination of distance from the radiation source position and received amount of ultraviolet radiation, than a majority, preferably all, of the plurality of space portions positioned in the space. Hence, in a yet further embodiment, for a majority, preferably all, space portions in the space said combination is higher than for the target space portion.

In an embodiment, the combination is a substantially linear, e.g. linear, combination, such as the sum or an average, of the, preferably normalised, distance from the radiation source position and the, preferably normalised, received amount of ultraviolet radiation. Experiments showed that such a combination yields satisfying results. The substantially linear combination may e.g. be a weighted average. The normalised distance from the radiation source position and the normalised received amount of ultraviolet radiation, preferably have substantially equal weights. Preferably, association of said combination with the target space portion, or with any other space portion, is established by means of a data structure in a memory of the controller, wherein said combination is linked to the respective space portion.

In an embodiment, the combination of distance from the radiation source position and received amount of ultraviolet radiation is a combination of a normalised distance from the radiation source position and a normalised received amount of ultraviolet radiation.

In an embodiment, the controller is arranged for determining, before the radiation source has reached the target position, an updated target position based on an update of the sensor signal and/or on an update of the cumulative exposure pattern. Preferably, the controller is arranged for controlling, before the radiation source has reached the target position, the movement means for moving the radiation source towards the updated target position. In this embodiment it may be prevented that the radiation source, and possibly the movement means and/or a housing of the robot, may cover the target position. Such coverage may diminish an intensity of the ultraviolet radiation that reaches the target position.

In an embodiment, the controller is provided with spatial information that relates to a configuration of the space. Preferably, the controller is arranged for determining the target position to extend the path in the space further based on the spatial information. Optionally, in use, the spatial information restricts the number of positions in the space from which the target position can be selected. The spatial information for example includes a plurality of positions in the space that are to be passed by the radiation source. The spatial information preferably includes a starting position of the radiation source. Thus, optionally, the controller is arranged for controlling the movement means for moving the radiation source along a path in the space that includes positions determined by the spatial information. Preferably, the spatial information includes a plurality of positions in the space that cannot be reached by the radiation source. Such plurality of positions are for example determined during a previous disinfection of the space.

In an embodiment, the spatial information includes a position of an object in the space and/or a boundary of the space. Preferably, the sensor means are arranged for generating a sensor signal that is indicative for a position of the object in the space and/or the boundary of the space. Preferably, the controller is arranged for determining the target position to extend the path in the space further based on the spatial information by preventing overlap between, on the one hand, the position of the radiation source and/or the target position, and, on the other hand, the position of the object in the space and/or the boundary of the space. In such an embodiment, mechanical contact between the radiation source may be prevented, or at least a chance of such contact may be decreased.

In an embodiment, the sensor includes a plurality of sensor devices. Preferably, one or more of the sensor devices are arranged to move together with the radiation source, for example are attached to the radiation source. Preferably, one or more of the sensor devices are arranged to not move together with the radiation source, in particular be stationary during movement of the radiation source. Preferably, the one or more sensor devices that do not move together with the radiation source, e.g. are stationary during movement of the radiation source, are communicatively connected to the controller by means a wireless communicative connection. The wireless communicative connection may be provided via an external computer, such as a server computer, to which the robot is communicatively connected.

In an embodiment, the sensor means include one or more, preferably a plurality of, ultraviolet radiation detector devices that are located at one or more, preferably at a plurality of, locations in the space. Preferably, the sensor signal includes a signal that is indicative for an amount of ultraviolet radiation received at the one ore more, preferably the plurality of, locations in the space. The controller may be arranged for determining the cumulative exposure pattern of the emitted ultraviolet radiation in the space, based on the signal that is indicative for an amount of ultraviolet radiation received at the one ore more, preferably the plurality of, locations in the space. Such determining may be in combination with, or alternative to, determining the cumulative exposure pattern of the emitted ultraviolet radiation in the space based on the path, on a duration and/or velocity of movement of the radiation source along the path, and on an intensity and/or direction of the ultraviolet radiation emitted during movement along the path.

In an embodiment, the controller is arranged for mapping the space by determining, based on the sensor signal, a distance to the object in the space and/or the boundary of the space. The spatial information preferably includes a map of the space.

In an embodiment, the map includes positions of objects in the space and/or boundaries of the space. The map preferably includes a position of a plurality of space portions in the space. Preferably, the map includes a temperature in the space, a humidity in the space, an intensity of use in the space, and/or an amount of dust in the space, associated with a position in the space and/or a space portion in the space. Preferably, a minimum exposure threshold is higher for positions in the space and/or space portions in the space that are associated with a higher temperature, a higher humidity, a higher intensity of use, and/or a higher amount of dust. After all, such places may be more vulnerable for pathogens to accumulate or grow. Also, the disinfection by ultraviolet radiation could be negatively influenced by, e.g., dust or humidity.

In an embodiment, the controller is arranged for determining the cumulative exposure pattern of the emitted ultraviolet radiation in the space further based on the spatial information and/or the disinfection information, for example on a humidity in the space and/or an amount of dust in the space. The controller may for example be arranged for determining a lowered exposure to ultraviolet radiation for positions in the space and/or space portions in the space, or spaces, that are associated with a higher humidity and/or a higher amount of dust. Optionally, the minimum exposure threshold is uniform in the space.

The spatial information can be provided to the controller before disinfecting starts. Optionally, the spatial information is obtained by the robot autonomously, by means of the sensor means and the controller. For example, the information on a position of an object in the space and/or a boundary of the space may be determined by the disinfection robot, in particular by means of the sensor means and the controller. The robot may e.g. be arranged for moving through the space autonomously, determining the information on a position of an object and/or of the boundary. Optionally, the spatial information is provided to the robot by a user of the robot or may be provided from a source that is external to the robot, such as a user or a server computer to which the robot is communicatively connected.

In an embodiment, the controller is arranged for using the spatial information for determining the cumulative exposure pattern. The information on an object in the space and/or a boundary of the space may e.g. be used for determining a shadow and/or reflection from the object and/or the boundary. In this way, a determination of the cumulative exposure pattern may be more accurate. Preferably, the controller is arranged for using a ray tracing algorithm when using the spatial information for determining the cumulative exposure pattern.

The controller may be arranged for determining the spatial information and/or the disinfection information, such as the amount of pathogen in the space, by means of the sensor means, in particular by means of the one or more sensor devices included by the sensor means. Alternatively, the spatial information and/or the disinfection information may optionally be received from an external source. In an embodiment, the controller is arranged for determining information included in the map by means of the one or more sensor devices included by the sensor means. In an embodiment, the sensor means are arranged for generating a sensor signal that is indicative for an amount of pathogen and/or allergen in the space, and/or for a type of pathogen and/or allergen in the space. The sensor means preferably include a sensor that is arranged for detecting an amount of pathogen and/or allergen in the space, and/or a sensor that is arranged for detecting a type of pathogen and/or allergen in the space.

Preferably, the controller is arranged for estimating spatial information and/or disinfection information in the space before starting disinfecting the space, based on spatial information and/or disinfection information obtained during prior disinfecting, e.g. based on one or more maps of the space obtained during prior disinfecting. Preferably, the spatial information and/or the disinfection information is associated with a time that is indicative for a time of generating the sensor signal on which the spatial information and/or the disinfection information is based. Preferably, the controller is provided with a map that is estimated prior to disinfecting.

In an embodiment, the controller is arranged for associating the cumulative exposure pattern to the object in the space and/or the boundary of the space. Optionally, such association is established by means of a data structure in a memory of the controller, wherein the cumulative exposure pattern is linked to the object in the space and/or the boundary of the space. In this way, the object and/or boundary can be used for determining the cumulative exposure pattern, for example when determining an amount of ultraviolet radiation received by a space portion that is in a shadow caused by the object or receives ultraviolet radiation reflected from the boundary. Preferably, the cumulative exposure pattern includes a plurality of space portions that are associated with a position of the space portion in the space, with an amount of ultraviolet radiation received in the space portion as a result of the disinfecting, and with information on an object and/or boundary being present in the space portion. For example, a two-dimensional grid or a three-dimensional grid in the space may be defined in the space, wherein grid elements of the grid are associated with the presence or absence of the object and/or boundary.

In an embodiment, the sensor means include one or more cameras for determining a position of an object in the space and/or a boundary of the space. The one or more cameras may include one or more two-dimensional cameras that, in use, yield a two-dimensional view of the space and/or one or more three-dimensional cameras that, in use, yield a three-dimensional view of the space. Thus, preferably, the sensor signal includes a signal obtained from a three-dimensional camera.

Alternatively, or additionally, the sensor means may for example include one or more laser emitters and/or one or more sensors for receiving the emitted laser light. Sensor means that include one or more cameras and one or more laser emitters, enable a relatively reliable sensor signal. As a result, an indication of the position of the radiation source in the space may be relatively reliable, as well as the determination of a position of an object in the space and/or a boundary of the space. Preferably, the controller is arranged for determining the cumulative exposure pattern based on a signal obtained from the three-dimensional camera. Preferably, one or more cameras included by the sensor means are arranged for generating a sensor signal that is indicative for an intensity of use in the space. Thus, preferably, the sensor means include one or more cameras for determining an intensity of use in the space.

Preferably, the one or more cameras are stationary, e.g. during disinfecting.

In an embodiment, the sensor means are arranged for generating a sensor signal that is indicative for a humidity in the space, for a temperature in the space, for an amount of ozone in the space, and/or for an amount of dust in the space. The sensor means preferably include a sensor device that is arranged for detecting a humidity in the space, a sensor device that is arranged for detecting a temperature in the space, a sensor device that is arranged for detecting an amount of ozone in the space, and/or a sensor device that is arranged for detecting an amount of dust in the space. Preferably, the humidity is a relative humidity. Preferably, the amount of dust is a dust content in the space, Preferably, the amount of ozone is an ozone content in the space.

The controller preferably is arranged for controlling the movement means for moving the radiation source, in particular for controlling the movement means for controlling a duration and/or velocity of moving the radiation source, along the path in the space, based on the sensor signal that is indicative for a humidity in the space, for a temperature in the space, and/or for an amount of dust in the space. By determining a humidity, temperature, and/or amount of dust in the space, the disinfecting may be improved by adjusting the velocity and/or duration of the movement of the radiation source along the path.

In an embodiment, the controller is arranged for controlling the movement means for moving the radiation source, in particular for controlling the movement means for controlling a duration and/or velocity of moving the radiation source, along the path in the space, based on the sensor signal that is indicative for the amount of ozone in the space. In this way, a safety of use of the disinfection device may be improved. The controller preferably is arranged for controlling the radiation source, in particular for controlling an intensity of ultraviolet radiation emitted by the radiation source, based on the sensor signal that is indicative for the amount of ozone in the space. Preferably, the controller is arranged to decrease the velocity of the radiation source and/or to decrease the intensity of the ultraviolet radiation emitted by the radiation source, if the sensor signal that is indicative for the amount of ozone in the space indicates an amount of ozone that is above a, preferably predetermined, ozone threshold. Preferably, the controller is arranged to halt emitting ultraviolet radiation by means of the radiation source, if the sensor signal that is indicative for the amount of ozone in the space indicates an amount of ozone that is above the ozone threshold. Optionally, the controller is arranged for turning the radiation source off. Preferably, the robot is arranged to generate a warning and/or to generate a recommendation to bring in fresh air into the space, if the amount of ozone in the space is above the ozone threshold.

Optionally, the humidity in the space, the temperature in the space, the amount of ozone in the space, and/or the amount of dust in the space is determined at least prior to disinfecting. Optionally, the humidity in the space, the temperature in the space, the amount of ozone in the space, and/or the amount of dust in the space is determined during and/or after disinfecting.

In an embodiment, the sensor means are arranged for generating a sensor signal that is indicative for a type of pathogen and/or allergen in the space, and/or for an amount of pathogen and/or allergen, for example a pathogen density and/or allergen density, in the space, in particular on a surface in the space and/or in air in the space. The sensor means preferably include a sensor device that is arranged for detecting a type and/or an amount of pathogen and/or allergen. The sensor means preferably include a sensor device for determining a type of pathogen and/or allergen, and/or for determining an amount of pathogen and/or allergen, for example based on a photonic method. The controller preferably is arranged for controlling the movement means for moving the radiation source, in particular for controlling the movement means for controlling a duration and/or velocity of moving the radiation source, along the path in the space, based on the sensor signal that is indicative for a type of pathogen and/or allergen in the space and/or for an amount of pathogen and/or allergen in the space. By determining a type and/or amount of pathogen and/or allergen, the disinfecting may be improved by adjusting the velocity and/or duration of the movement of the radiation source along the path.

In an embodiment, the controller is provided with disinfection information that relates to a quality of the disinfection, wherein the controller is arranged for determining the target position to extend the path further based on the disinfection information. Taking into account disinfection information, may improve the quality of the disinfection of the space. Preferably, the controller is arranged for storing and/or receiving disinfection information. Preferably, the robot includes a communication device that is arranged for receiving the disinfection information. Preferably, the disinfection information includes the humidity in the space, the temperature in the space, the amount of ozone in the space, and/or the amount of dust in the space. Preferably, such elements of the disinfection information are obtained or received by the robot prior to disinfecting the space.

In an embodiment, the disinfection information includes a, preferably predetermined, minimum exposure threshold and/or an, optionally predetermined, minimum part of the space that is to reach the minimum exposure threshold. Preferably, the controller is arranged for determining the target position to extend the path further based on the disinfection information by extending the path at least until the amount of received ultraviolet radiation of the cumulative exposure pattern is above the minimum exposure threshold in at least the minimum part of the space.

Such an embodiment enables finding a balance between disinfection quality and the time needed for disinfecting the space. After all, disinfecting may be stopped after the minimum exposure threshold has been reached for only the minimum part of the space.

The minimum part of the space may for example be a numerical value, such as 50% of the space, 75% of the space or 90% of the space. Alternatively, or additionally, the minimum part of the space preferably is determined by an intensity of use in the space. Such an intensity of use may e.g. be determined by an occupation rate, or frequency of occupation, of portions of the space by one or more persons in the space. Preferably, in the minimum part of the space, the intensity of use is above a, preferably predetermined, minimum use threshold. Preferably, outside the minimum part of the space, the intensity of use is below the minimum use threshold. Preferably, the disinfection information includes the minimum use threshold and/or the intensity of use in the space. Preferably, such elements of the disinfection information are obtained or received by the robot prior to disinfecting the space. In another example, the minimum part of the space may be occupied by an object in the space and/or a boundary of the space. Preferably, the controller is arranged for extending the path at least until an amount of ultraviolet radiation in the cumulative exposure pattern is above a minimum exposure threshold for the object and/or the boundary, preferably for a majority of the objects and/or boundaries, more preferably for all objects and/or boundaries. In such an embodiment, the controller may determine, based on the minimum exposure threshold, whether an object and/or boundary has received a sufficient amount of ultraviolet radiation.

In an embodiment, the cumulative exposure pattern includes a plurality of space portions that are associated with a space portion position in the space, an amount of ultraviolet radiation received in the space portion, and a surface property in the space portion. Preferably, the minimum exposure threshold is based on the surface property. In this way, the controller may adapt the amount of ultraviolet radiation received by a space portion, to a surface property of the space portion. The surface property may for example be determined by a surface of an object in the space and/or by a surface of a boundary of the space. A surface property may e.g. be an orientation of the surface and/or a material of the surface. The orientation of the surface preferably includes a tilt or an inclination of the surface, e.g. a tilt or an inclination relative to a horizontal or vertical direction. Alternatively, or additionally, a surface property may e.g. be a reflectivity, a porosity, a structure, and/or a shadow effect on the surface. A surface that is positioned so that, during disinfecting, a shadow effect on that surface is relatively strong, may need more direct ultraviolet radiation than a surface on which such a shadow effect is relatively weak. A shadow effect on a surface may be relatively strong for example if the surface faces a boundary of the space close to the boundary, or if the surface is closely surrounded by objects in the space. The controller may be arranged to infer from the sensor means, in particular a camera of the sensor means, one or more surface properties in the space.

In an embodiment, the disinfection information includes information on a pathogen type, allergen type, and/or required level of disinfection. The pathogen type may for example be determined by a morphology of the pathogen. The allergen type may for example be determined by a morphology of the allergen. Preferably, the disinfection information includes a pathogen and/or allergen density in the space, in particular on a surface in the space and/or in air in the space. Preferably, the controller is arranged for determining the target position to extend the path further based on the disinfection information by determining the duration and/or velocity of movement of the robot, in particular of the radiation source, along the path based on the information about the pathogen type, allergen type, and/or required level of disinfection. In such an embodiment, the disinfection can be directed to a specific pathogen type and/or allergen type. At the same time, a duration and/or velocity of the disinfection can be directed to the required level of disinfection. For example, the level of disinfection is that at least 99%, preferably at least 99.9%, of all pathogens and/or allergens in the space are inactivated.

The disinfection information may additionally, or alternatively, include various types of other information that may be taken into account for disinfecting. For example, the disinfection information may include information on an intensity of the radiation source, e.g. at a specified distance of the radiation source, and/or information on a radiation power of the radiation source. Optionally, the disinfection information includes the range from the target space portion that may be used for determining the target space portion.

In an embodiment, the controller includes a global planner for taking into account the spatial information and a local planner for determining the target position. Preferably, the local planner is arranged to receive the sensor signal that is indicative for a position of the radiation source in the space. Preferably, the global planner is arranged for influencing the determination of the target position by the local planner, based on the spatial information.

Preferably, the global planner and/or the local planner are arranged for storing and/or receiving disinfection information. Preferably, the local planner is arranged for determining the target position to extend the path in the space further based on the disinfection information. Preferably, the global planner is arranged for influencing the determination of the target position to extend the path in the space further based on the disinfection information.

Preferably, the global planner and/or the local planner is provided with information on a position of an object in the space and/or a boundary of the space. Preferably, the local planner is arranged for determining the target position to extend the path in the space further based on the position of the object in the space and/or the boundary of the space. Preferably, the global planner is arranged for influencing the determination of the target position to extend the path in the space further based on the position of the object in the space and/or the boundary of the space. In an embodiment, the global planner is provided with the map of the space, preferably with the estimated map. Preferably, the global planner is provided with the map of the space before starting disinfecting. Preferably, the controller is arranged for updating the map, preferably during disinfecting, based on the sensor signal.

In an embodiment, the information on a position of an object in the space and/or a boundary of the space, includes a distance from the radiation source to an object in the space and/or a boundary of the space. In an embodiment, the local planner is arranged for determining the target position to extend the path in the space further based on a sensor signal that is indicative of a distance to an object in the space and/or a boundary of the space. Preferably, the sensor means are arranged to determine the distance to an object in the space and/or a boundary of the space by triangulation of emitted laser light that is reflected from said object or boundary, by determining a time of flight of the laser light after reflection, and/or by determining a phase shift. The sensor means preferably include one or more laser emitters and one or more laser detectors for receiving the emitted laser light. Preferably, the sensor means include one or more cameras for determining the distance from the radiation source to the position of an object in the space and/or a boundary of the space. In an embodiment, the disinfection robot includes a communication device for sending information to the robot and/or receiving information from the robot. The communication device preferably includes an antenna. The communication device preferably is arranged for sending information to the robot and/or for receiving information from the robot. Preferably, the controller is arranged for generating a disinfection report that contains a result of the disinfection and further contains disinfection information and/or spatial information used during disinfection. The robot preferably is arranged for sending the disinfection report, e.g. to a user and/or to an external computer such as a server computer.

The invention also provides a system that includes a disinfection robot according to the invention and a remote computer, wherein the remote computer preferably is communicatively connected to the robot for receiving information from the robot and/or sending information to the robot. Preferably, the remote computer is communicatively connected to a plurality of robots according to the invention. The invention further provides a method of disinfecting a, preferably indoor, space such as a room of a hospital or a nursing centre, by means of ultraviolet radiation that is emitted in the space by means of a radiation source that is included by a disinfection robot for disinfecting the space, the method including: generating, by means of sensor means that are included by the robot, a sensor signal that is indicative for a position of the radiation source in the space, and receiving, by means of a controller that is included by the robot and is communicatively connected to the sensor means, the sensor signal; and/or moving the radiation source in the space by means of movement means that are included by the disinfection robot, and controlling, by means of the controller, said moving along a path in the space. Said path may be formed by a sequence of the positions of the radiation source in the space.

According to an aspect, the method includes determining, by means of the controller, a cumulative exposure pattern of the emitted ultraviolet radiation in the space preferably based on the path, on a duration and/or velocity of movement of the radiation source along the path, and/or on an intensity and/or direction of the ultraviolet radiation emitted during movement along the path. According to another aspect, the method includes determining, by means of the controller, a target position to extend the path in the space based on the sensor signal and on the cumulative exposure pattern in the space, and controlling the movement means for moving the radiation source towards the target position.

Determining the target position based on the sensor signal and the cumulative exposure pattern, enables the controller to set a target position for the radiation source at a position where an improvement of the cumulative exposure pattern can be reached at a relatively short distance. As a result, the space can be disinfected in an acceptable time and to a sufficient extent. After all, the cumulative exposure pattern indicates which portions of the space were exposed to the ultraviolet radiation and indicates the total amount of ultraviolet radiation that was received as a result of the disinfection.

In an embodiment, the cumulative exposure pattern includes a plurality of space portions that are associated with a position of the space portion in the space and with an amount of ultraviolet radiation received in the space portion. Preferably, the method includes: determining, by means of the controller, the target position in a target space portion of the plurality of space portions, based on a combination, in the target space portion, of distance from the radiation source position and received amount of ultraviolet radiation. Preferably, the method includes: determining, by means of the controller, the target position in a target space portion of the plurality of space portions that is associated with a lower combination of distance from the radiation source position and received amount of ultraviolet radiation, than another space portion in the space, preferably lower than another space portion positioned in a, preferably predetermined, range from said target space portion, more preferably lower than another space portion positioned adjacent to the target space portion.

In an embodiment, the method includes: determining the target position in a target space portion of the plurality of space portions that is associated with a lower combination of distance from the radiation source position and received amount of ultraviolet radiation, than a majority, preferably all, of the space portions adjacent to the target space portion, preferably lower than a majority, preferably all, of the space portions positioned in a, preferably predetermined, range from the target space portion, more preferably lower than a majority, preferably all, of the plurality of space portions in the space.

It may thus be clear that, in an embodiment, the method includes: determining the target position in a target space portion of the plurality of space portions that is associated with a lower combination of distance from the radiation source position and received amount of ultraviolet radiation, than a majority, preferably all, of the plurality of space portions in the space and/or of the space portions in a range from the target space portion. It may further be clear that, in an embodiment, the method includes: determining the target position in a target space portion of the plurality of space portions that is associated, in a range from the target space portion and/or adjacent to the target space portion, with a minimum of a combination of distance from the radiation source position and received amount of ultraviolet radiation.

In an embodiment, the combination is a substantially linear, e.g. linear, combination, such as the sum or an average, of the, preferably normalised, distance from the radiation source position and the, preferably normalised, received amount of ultraviolet radiation.

In an embodiment, the combination of distance from the radiation source position and received amount of ultraviolet radiation is a combination of a normalised distance from the radiation source position and a normalised received amount of ultraviolet radiation.

The invention further provides a computer program product that includes instructions that, when carried out by a processor of a controller, carry out a step of a method according to the invention, which step includes determining, preferably by means of the controller, the target position to extend the path in the space based on the sensor signal and on the cumulative exposure pattern in the space.

The invention further provides a computer-readable storage medium that includes instructions that, when carried out by a processor of a controller, carry out a step of a method according to the invention, which step includes determining the target position to extend the path in the space based on the sensor signal and on the cumulative exposure pattern in the space. The storage medium preferably is formed by a memory of the controller.

The invention further provides use of a disinfection robot according to the invention and/or a system according to the invention. Preferably, the robot and/or system is used in a method according to the invention. The robot preferably is arranged for carrying out a method according to the invention. The robot preferably includes the computer program product, or another computer program product, and/or the computer-readable storage medium.

The invention also provides a data structure. The data structure preferably is suitable for use in disinfecting a, preferably indoor, space such as a room of a hospital or a nursing centre, by means of ultraviolet radiation. The data structure preferably is stored in a memory of a robot, in particular of a controller of the robot. Preferably, in the data structure, a plurality of space portions are defined and individually linked to a position in the space of the space portion, to a received amount of ultraviolet radiation in the space portion, and/or to a surface property in the space portion. Preferably, in the data structure, the plurality of space portions are also linked, individually or collectively, to a humidity in the space, to a temperature in the space, to an amount of ozone in the space, and/or to an amount of dust in the space. Preferably, the data structure also includes a sensor time associated with a time that is indicative for a time of generating the sensor signal on which an element of the data structure, such as temperature or humidity, is based. Preferably, in the data structure, the plurality of space portions are also linked to an amount of ultraviolet radiation received by one or more ultraviolet radiation detector devices, to a type of pathogen and/or allergen, and/or to an amount of pathogen and/or allergen. Preferably, in the data structure, the plurality of space portions are also linked, individually or collectively, to an intensity of use in the space and/or to a minimum use threshold. Preferably, in the data structure, the plurality of space portions are also linked, individually or collectively, to a minimum exposure threshold and/or to an ozone threshold. In an embodiment, the data structure is included in, or forms, a cumulative exposure pattern, e.g. a cumulative exposure pattern as described herein.

Optionally, the received amount of ultraviolet radiation included in the data structure is determined by means of the controller based on the path, on a duration and/or velocity of movement of the radiation source along the path, and on an intensity and/or direction of the ultraviolet radiation emitted during movement along the path. Alternatively, or additionally, the received amount of ultraviolet radiation included in the data structure optionally is determined by means of the one or more ultraviolet radiation detector devices. Preferably, both ways of determining the received amount of ultraviolet radiation are combined. For example, the amount of ultraviolet radiation detected by a detector device may be used to calibrate the received amount of ultraviolet radiation determined by means of the controller based on the path, on the duration and/or velocity of movement of the radiation source along the path, and on the intensity and/or direction of the ultraviolet radiation emitted during movement along the path.

Preferably, in the data structure, the position in the space is linked with the presence or absence of an object in the space and/or a boundary of the space. In an embodiment, the data structure is included in, or forms, a map of the space. Preferably, in the data structure, a combination of distance from a radiation source position and a received amount of ultraviolet radiation, e.g. a combination described herein, is linked with the position of the individual space portions. In an embodiment, by means of the data structure, the cumulative exposure pattern is linked to an object in the space and/or a boundary of the space.

Preferably, the data structure is based on a sensor signal that is indicative for a position of the radiation source in the space. In an embodiment, the data structure is used by a controller that is arranged for determining a target position to extend a path in the space based on the sensor signal and on the cumulative exposure pattern in the space, and for controlling the movement means for moving the radiation source towards the target position. The data structure preferably is used in a robot and/or a method according to the invention.

The invention also provides a data structure product that includes the data structure and a computer-readable medium that includes the data structure.

### BRIEF DESCRIPTION OF THE DRAWINGS

The invention will be illustrated with reference to the following non-limiting figures, wherein:

FIG. 1 shows a schematic view of parts that may be included by a robot, in an embodiment of the invention;

FIG. 2A shows a schematic side view of a device in an embodiment of the invention;

FIG. 2B shows another schematic side view of the device of FIG. 2A, in a direction that is orthogonal to the direction of FIG. 2A;

FIG. 2C shows a schematic top view of the device of FIG. 2A;

FIG. 2D shows a schematic bottom view of the device of FIG. 2A;

FIG. 3 shows an example of irradiance generated by a radiation source;

FIG. 4A shows an example of a normalised distance from a radiation source position to a space portion at a certain moment in time;

FIG. 4B shows an example of a normalised cumulative exposure pattern;

FIG. 4C shows the sum of a normalised distance from the radiation source position and a normalised received amount of ultraviolet radiation, divided by two;

FIG. 5A schematically illustrates an example of a path in a space;

FIG. 5B schematically illustrates a robot in an embodiment of the invention;

FIG. 6 schematically illustrates a method of disinfecting, in an embodiment of the invention;

FIG. 7 schematically shows a system that includes a robot; and

FIG. 8 shows an example of a disinfection report.

### DETAILED DESCRIPTION

Embodiments of the invention that may be illustrated with reference to one or more of the figures, may relate to a, partly or completely autonomous, robot 2 that is arranged for disinfecting a space 32, preferably an indoor space. The robot 2 may be provided with sensor means 8, in order to determine the surroundings of the robot 2 or a part thereof. The robot 2 may include a radiation source 4 for emitting ultraviolet radiation (also referred to as UV radiation). The robot may further include a controller 10 that is arranged to control movement means 6 of the robot 2 in order to move the radiation source 4 in the space 32 that is to be disinfected. The controller 10 of the robot 2 may be arranged for setting a target position and moving the radiation source 4 towards the target position. Thereto, the controller may include a processor 10.1 and memory 10.2. An algorithm that is implemented in a computer program that, in use, may run on the controller 10, may be used for determining the target position. The controller may control the velocity of the movement, to sufficiently disinfect the space 32 against a pathogen type and/or an allergen type.

In embodiments of the invention, the radiation source 4 may be designed to provide germicidal ultraviolet radiation for disinfecting air and/or surfaces in the space 32. The space 32 may be bounded by one or more boundaries 38, such as a wall, a door or a window. The space 32 may be empty or may contain one or more objects 42 such as a chair, table, closet or bed. Disinfecting both air and surfaces brings the advantage that deposition of active pathogens and/or allergens on previously disinfected surfaces, can be diminished. Disinfection generally may prevent volumes and surfaces from containing active microorganisms like bacteria and viruses. During disinfection, the ultraviolet disinfection dose generally may be a function of exposure time to the ultraviolet radiation, of distance to an ultraviolet radiation source of the robot 2, and of the radiant power of the radiation source 4.

In embodiments of the invention, the controller may be arranged to generate a two-dimensional or three-dimensional map of the space based on a sensor signal generated by the sensor means 8. The controller may regard the space to vary in only two dimensions or in three dimensions. The controller 10 may be arranged for defining a grid in the space 32. The map may include the grid. The map and/or the grid may be stored in the memory 10.2 of the controller 10. The robot may further be arranged for determining dimensions of the space 32 to be disinfected and a position of one or more obstructing objects and/or or boundaries. The robot 2 may be arranged for associating one or more elements of the grid with one or more objects and/or boundaries. A grid element may be associated with a position of the radiation source. Association may e.g. be established by means of a data structure in the memory 10.2, wherein the grid element is linked to the position of the radiation source.

In embodiments of the invention, disinfection information 68 and/or spatial information 64 with respect to the space 32 may be obtained from the memory 10.2 and/or may be received from an external source. The controller of the robot may be arranged for determining a path in the space, based on the disinfection information and/or spatial information, such as the map of the space 32. The controller 10 of the robot 2 may be arranged for determining a velocity of the robot 2 during disinfecting. In this way, it may be achieved that pathogens and/or allergens have received a sufficient amount of ultraviolet radiation, given the selected pathogens and/or allergens against which the space is to be disinfected.

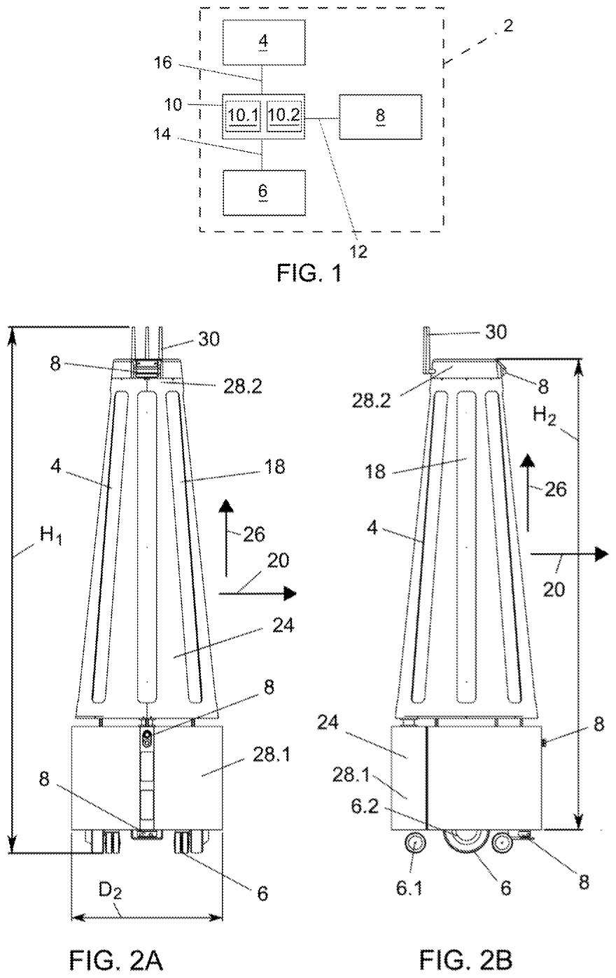

FIG. 1 shows a schematic representation of a robot 2, in an embodiment of the invention. The robot is arranged for disinfecting a, preferably indoor, space such as a room of a hospital or a nursing centre, by means of ultraviolet radiation. The disinfection robot is arranged to include, and preferably includes, a radiation source 4. The radiation source 4 is arranged for emitting the ultraviolet radiation. The disinfection robot also includes movement means 6 for moving the radiation source in the space. The radiation source further includes sensor means 8 for generating a sensor signal that is indicative for a position of the radiation source 4 in the space. Sensor signals that contain information from which the position of the radiation source can be inferred or estimated, may generally be considered indicative for said position, and vice versa.

The robot 2 further includes a controller 10 that, more in general, is arranged for receiving the sensor signal. The controller may be communicatively connected to the sensor means 8 for receiving the sensor signal. The communicative connection 12 between the sensor means and the controller may be wired and/or wireless. The sensor signal may for example be an electronic signal. In a particular variation, the sensor signal may be a mechanical signal. Such a mechanical signal may be transmitted by a moveable part of the robot 2.

The controller 10 is arranged for controlling the movement means for moving the radiation source along a path in the space. The path is formed by a sequence of the positions of the radiation source in the space. The path may be determined, or may at least be influenced, by a sequence of target positions. The controller may also be communicatively connected to the movement means, for sending to the movement means a movement signal. The communicative connection 14 between the controller and the movement means 6 may be wired and/or wireless.

The controller 10 may further be arranged for controlling the radiation source 4. The controller, or at least a part thereof, may be communicatively connected with the radiation source 4. The communicative connection 16 between the controller 10 and the radiation source 4 may be wired and/or wireless. A wireless connection may enable the radiation source to move freely with respect to the controller 10, or at least a part of the controller 10. Controlling the radiation source may include turning the radiation source on or off. When the radiation source moves through the space radiating ultraviolet radiation, the controller may store in its memory the intensity, e.g. a level of the radiant energy, emitted by the radiation source and/or the position of the radiation source in the space.

The controller is further arranged for determining a cumulative exposure pattern of the emitted ultraviolet radiation in the space based on the path. The cumulative exposure pattern may be formed by a data structure or by a signal that is indicative for a received amount of ultraviolet radiation as a result of the disinfecting dependent on a position in the space. Said determining may be based on a duration and/or velocity of movement of the radiation source along the path, and on an intensity and/or direction of the ultraviolet radiation emitted during movement along the path. The controller may be provided with a computer program product that includes instructions that are designed to calculate the cumulative exposure pattern.

The calculation may be based on, optionally predetermined, information provided to the controller from an external source and/or on information measured by means of the sensor means, prior to disinfecting or during disinfecting.

The controller 10 may include a processor 10.1 and a memory 10.2. The controller 10 is arranged for determining a target position to extend the path in the space based on the sensor signal and on the cumulative exposure pattern in the space. In this way, the controller may control the extension of the path to positions where an improvement of the cumulative exposure pattern can be reached and/or that are relatively close to the current position. The controller may further be arranged for controlling the movement means for moving the radiation source towards the target position.

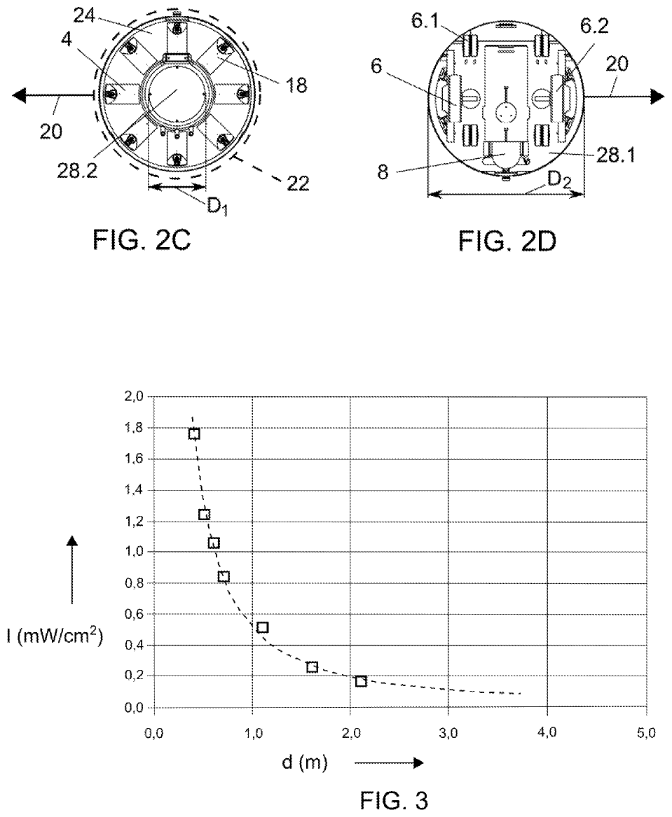

FIGS. 2A and 2B show, in schematic side views from mutually orthogonal directions, a robot 2 in an embodiment of the invention. FIGS. 2C and 2D show a schematic top view respectively bottom view of the robot 2 in an embodiment of the invention. The disinfection robot 2 may be arranged for disinfecting an indoor space such as a room, a corridor, a hall, or a part thereof. The disinfection robot may in particular be arranged for disinfecting an indoor space of a hospital or a nursing centre. The indoor space may be a room of a hospital or nursing centre that is used for treating a patient, after the disinfecting.

The disinfection robot 2 is arranged for disinfection by using ultraviolet radiation. Thereto, the disinfection robot is arranged to include, and preferably includes, a radiation source 4 that is arranged for emitting the ultraviolet radiation. The radiation source 4 may include a plurality of lamps 18 that, in use, emit ultraviolet radiation. The lamps may have a longitudinal shape that, in use, extends upwards. For lamps that have a longitudinal shape and extend upwards (or downwards), a robot having at least four, preferably at least six, more preferably at least eight, lamps enables a relatively uniform emittance of ultraviolet radiation in a lateral direction 20 along an outer circumference 22 of the radiation source 4. Less than four, for example one, two or three, lamps 18 may be used alternatively. FIGS. 2A-2C show a radiation source provided with eight lamps having a longitudinal shape that extends upwards. Preferably, all lamps 18 are provided to the robot 2 at the same time. Preferably, the number of burning hours may be approximately similar for all lamps of the plurality of lamps 18.

The ultraviolet radiation may be in a range between 100 and 400 nanometre, preferably between 100 and 280 nanometre (also referred to as UV-C radiation). Many types of pathogens and allergens are susceptible to inactivation through exposure to UV-C radiation. UV-C radiation may be absorbed by RNA and DNA molecules, and may induce changes in their structure. This may result in their inability to replicate, so that they are inactivated. In particular, the emitted ultraviolet radiation may be in a, preferably narrow, spectrum that peaks at or around a wavelength of 254 nanometre. This wavelength is effective against many microorganisms including many types of viruses, bacteria, and mold spores. In another embodiment, the peak of the emitted spectrum may be at a different effective wavelength in the ultraviolet range. Lamps of various types can be included by the radiation source. Disinfection can for example be reached by using eight lamps of the type Philips TUV 75W HO 1SL/6, with a nominal UV-C radiant power of 25.5 W per lamp. Using eight of such lamps may yield a total UV-C radiant power Φ of 180 W (this may represent lamp radiant power only and not include ballast loss).

The disinfection robot 2 may generally include a housing 24. The housing may be provided with the radiation source 4, optionally including the lamps 18. Alternatively, the radiation source 4, or a part thereof, may be separate from the housing 24. For example, the ultraviolet lamps may be packed separately from the housing during transport of the robot. In that way, the lamps can be protected safely. Germicidal ultraviolet lamps are relatively vulnerable when subjected to various kinds of mechanical loading. The lamps 18 may be reinstalled at a later moment, before disinfecting the space. Part of the radiation source, such as wiring, may be provided in an enclosure formed by the housing. Other wiring and/or electronic parts, such as the controller 10, may also be housed in the enclosure formed by the housing 24. The housing 24 may for example include stainless steel and/or anodized aluminium plates. Such a housing 24 may provide strength and durability in order to withstand all kinds of environments. Alternatively, or additionally, other durable and strong materials may be used to form the housing 24. A weight of the robot 2 may be in a range from 50 kg to 150 kg, for example around 90 kg.

A height H₁ of the robot 2, and/or a height H₂ of the housing 24, may be in a range from 1 m to 2.5 m, for example a height H₁ of the robot 2 of approximately 1.7 m and a height H₂ of the robot 2 of approximately 1.5 m. A diameter D₁ of a lower part 28.1 of the robot 2, and/or a diameter of a lower part of the housing 24, may be in a range from 0.40 m to 0.60 m, for example approximately 0.53 m. A diameter D₂ of an upper part 28.2 of the robot 2, and/or a diameter of an upper part of the housing 24, may be in a range from 0.20 m to 0.40 m, for example approximately 0.28 m. Such dimensions may enable a robot that is robust and has a substantial outer surface for radiating ultraviolet radiation, while the robot may still reach relatively narrow parts of the space.

More in general, the robot and/or the housing may be tapered in an, in use, upwards direction 26. A lateral (e.g. horizontal) dimension of the housing may decrease when going in the upwards direction 26. As a result of such a tapering or decrease, the lamps 18 may, in use, be inclined relative to the vertical direction. As a result, ultraviolet radiation may be directed to parts of the space that are located higher than the radiation source. In this way, reflections against a ceiling of the space may be established. Such reflections may increase the amount of radiation in parts of the space that are in a shadow of the ultraviolet radiation, e.g. caused by an object in the space.

The disinfection robot also includes the movement means 6 for moving the radiation source 4 in the indoor space. The movement means 6 may include a plurality of wheels 6. The plurality of wheels may include one or more wheels 6.2 that are steerable, preferably by being controlled by the controller 10 of the robot. The plurality of wheels may include one or more wheels 6.1 that are not steerable. Driving one or more of the wheels may be controlled by the controller. In this way, the controller may control a velocity, and hence a duration, of movement of the radiation source along the path in the space. The rotation and/or direction of the wheels 6 may, in use, be controlled by the controller of the robot. In other examples, the robot is provided with other movement means, such as small caterpillar tracks. The movement means may be formed by various types of moveable base that are suitable for moving the radiation source and for control of its velocity and direction.

The robot 2 also includes sensor means 8 for generating a sensor signal that is indicative for a position of the radiation source in the indoor space. Thus, the sensor signal may contain information from which the position of the radiation source can be inferred or estimated. The sensor means may be arranged for scanning the surroundings of the robot and/or the radiation source and determining distances between the radiation source of the robot and one or more objects and/or boundaries. The sensor means may e.g. include one or more laser emitters and one or more laser detectors for receiving the emitted laser light. A distance to an object in the space or boundary of the space may e.g. be determined by triangulation of emitted laser light that is reflected from said object or boundary, by determining a time of flight of the laser light after reflection (Lidar), and/or by determining a phase shift. Such ways of determining a distance are known as such to the skilled person.

The laser emitter, and optionally the laser detector, may be arranged to move, e.g. rotate, in order to scan the surroundings. Other sensor means may, alternatively or additionally, be used as well. The sensor means may e.g. include a sensor that measures rotation of a wheel of the robot and a magnetic sensor that measures a movement direction of the robot, in particular of the radiation source. The sensor means may include one or more motion sensors, for determining a position and/or velocity of the radiation source 4. The sensor means may include an inertial measurement unit, to determine an orientation and/or acceleration of the radiation source 4. The sensor signal may thus carry information on a position of the radiation source, that may enable to determine the position of the radiation source. The sensor signal may be arranged for basing thereon a, preferably three-dimensional, view of the surroundings of the robot and/or the radiation source.

The robot 2 further includes the controller 10 that is arranged for receiving the sensor signal. The controller may be positioned inside the housing 24. The controller 10 may be communicatively connected to the sensor means for receiving the sensor signal. The controller may e.g. include a processor and a memory, and may be arranged for running a computer program. The controlling by means of the controller may generally be carried out by means of the computer program. The controller may be arranged for controlling the movement means for moving the radiation source along the path in the space. In use, a velocity and/or direction of the radiation source may be controlled by the controller 10. The path may be formed by a sequence of the positions of the radiation source in the indoor space.

The controller may be further arranged for mapping the space by determining distances to one or more objects in the space and/or one or more boundaries of the space. Thus, the map may be formed by a collection of distances from the radiation source to one or more boundaries and/or objects in the space. In other variations, the map may include other and/or additional information such as the temperature, dust content, intensity of use, and/or humidity in the space. Thus, the mapping may optionally include determining a temperature, amount of dust, intensity of use, and/or humidity in the space. The map may generally represent a distribution of such variables in the space. Said mapping may be enabled by the sensor means 8, which may for example include one or more cameras for determining a position of an object in the space and/or a boundary of the space. The one or more cameras, optionally together with the controller, may be arranged for generating a two-dimensional or three-dimensional representation of the space. The sensor means 8 that are arranged for determining a position of the radiation source in the space, may optionally also be used for generating a sensor signal that is indicative for a position of the object in the space and/or the boundary of the space, and the other way round. Alternatively, or additionally, a map of the space that includes one or more objects in the space, one or more boundaries of the space, the temperature in the space, the dust content in the space, and/or the humidity in the space, may be provided to the controller 10. It may however be appreciated that the information on a position of one or more objects in the space, one or more boundaries of the space, the temperature in the space, the dust content in the space, and/or the humidity in the space may be obtained by the disinfection robot itself.

The robot may be provided with a communication device 30, such as an antenna for wireless communication. By means of the communication device 30, information can be exchanged with, i.e. can be sent to and/or received from, the robot. By means of the communication device, the information on a position of an object in the space and/or a boundary of the space, may, alternatively or additionally, be provided to the controller from an external source. Such an external source can be a user of the robot and/or a server computer to which the controller may be communicatively connected.

The controller 10 is arranged for determining a cumulative exposure pattern of the emitted ultraviolet radiation in the indoor space based on the path, i.e. based on the sequence of positions of the radiation source in the space during disinfection. The controller 10 is further arranged for determining the cumulative exposure pattern of the emitted ultraviolet radiation in the indoor space based on a duration and/or velocity of movement of the radiation source along the path, and on an intensity and/or direction of the ultraviolet radiation emitted during movement along the path.

The controller 10 may for example be arranged for storing a received amount of ultraviolet radiation for a plurality of space portions. This received amount of ultraviolet radiation may be used as input in an algorithm that is implemented in a controlling computer program that, in use, may run on the controller for determining the target position.

The received amount of ultraviolet radiation may for example be determined by the controller 10 by calculating a radiation intensity, such as irradiance, accumulated over time during disinfection of the space. The cumulative exposure pattern may represent, depending on a position in the space, the amount of radiation received as a result of the disinfection. Such a total amount may for example be determined by the controller 10 based on an estimated ultraviolet radiation intensity field that is present around the radiation source, the path of the radiation source, and a time of exposure. In such a calculation, the controller 10 preferably takes into account a decrease in the radiant power of the ultraviolet radiation that may occur during the lifetime of the radiation source. In this way, determining the exposure pattern takes into account a decreasing radiant power of the radiation source with time, as well as a decrease of the intensity with increasing distance from the radiation source.

In a variation of an embodiment described with reference to FIG. 1, and/or in an embodiment described with reference to FIGS. 2A-2D, space portions of the cumulative exposure pattern may further be associated with a surface property in the space portion, In this way, the controller 10 may adapt the amount of ultraviolet radiation to be received by a space portion, to a surface property of the space portion. The surface property may for example be determined by a surface of an object in the space and/or by a surface of a boundary of the space. A surface property may e.g. be an orientation of the surface and/or a material of the surface. Surfaces that are oriented substantially horizontally, may receive less ultraviolet radiation than surfaces that are oriented substantially vertically. Alternatively, or additionally, a surface property may be a reflectivity, a porosity, a structure, and/or a shadow effect on the surface. A relatively porous surface may need a relatively large amount of ultraviolet radiation. A surface that is positioned so that, during disinfecting, a shadow effect on that surface is relatively strong, may need more direct ultraviolet radiation than a surface on which such a shadow effect is relatively weak.

In said variation, the sensor signal may include signals that are indicative for different variables and are obtained from different sensor devices that may be included by the sensor means 8. The sensor means may include a sensor device that is arranged to be stationary during movement of the radiation source 4. The sensor device may be an ultraviolet radiation detector device, for example. The sensor device may be located in the space. The sensor signal may include a signal that is indicative for an amount of ultraviolet radiation received at the location of the ultraviolet radiation detector device. The controller 10 may be arranged for determining the cumulative exposure pattern further based on the signal that is indicative for the amount of ultraviolet radiation received at said location in the space. For example, the amount of ultraviolet radiation detected as the location of the ultraviolet radiation detector device, may be compared with the amount of ultraviolet radiation received at said location according to the calculated cumulative exposure pattern. Alternatively, or additionally, the controller may generally be arranged for calibrating the amount of ultraviolet radiation generated by the radiation source, by means of the ultraviolet radiation detector device.

In said variation, the sensor means 8 may be further arranged for generating a sensor signal that is indicative for a humidity in the space, for a temperature in the space, for an amount of ozone in the space, and/or for an amount of dust in the space. Thus, for example, the sensor signal may be indicative for a humidity in the space and for an amount of dust in the space. In another example, the sensor signal may be indicative for a humidity in the space, for a temperature in the space, and for an amount of dust in the space. The amount of dust in the space and/or the temperature in the space, may be determined using infrared radiation, for example. The dust may be formed by a wide range of types of particles. Sensor devices for determining the humidity, temperature, amount of ozone, and/or amount of dust in the space are known as such to the skilled person. Such sensor devices may move together with the radiation source 4, or may be stationary during movement of the radiation source 4. A wireless communicative connection between such sensor devices and the controller 10 may be provided, in particular when the sensor devices do not move together with the radiation source 4. The wireless communicative connection may be provided via an external computer, such as a server computer, to which the controller 10 of the robot 2 may be communicatively connected.

A quality of disinfecting the space may be influenced by the humidity, the temperature, and/or the dust content in the space. An ozone content in the space that is too high, may be harmful to humans and animals. This may be especially relevant, as the disinfecting may generate ozone in the space by means of the ultraviolet radiation. The controller 10 may be arranged to decrease the intensity of the ultraviolet radiation emitted by the radiation source 4, or halt emitting ultraviolet radiation by means of the radiation source 4, if the sensor signal that is indicative for the amount of ozone in the space indicates an amount of ozone that is above an ozone threshold. The robot 2 may be arranged to generate a warning and/or to generate a recommendation to bring in fresh air into the space, if the amount of ozone in the space is above the ozone threshold. By bringing fresh air into the space, an operator may improve safety of disinfecting. If the humidity is too high, the amount of dust in the space is too high, and/or a temperature in the space e.g. is too low, a quality of disinfecting may be negatively influenced. The controller 10 may be arranged for controlling a duration and/or velocity of moving the radiation source along the path in the space, based on the sensor signal that is indicative for the humidity in the space, for the temperature in the space, and/or for an amount of dust in the space. As a result, for a space that is relatively humid or is relatively dusty, a velocity of the radiation source may be controlled to be lower and/or a duration of the movement of the radiation source 4 may be controlled to be higher.

FIG. 3 shows an example of measured values (represented by squares) of irradiance I (in mW/cm²) generated by a radiation source being made of eight lamps of the type Philips TUV 75W HO 1SL/6, with a total UV-C radiant power Φ=180 W. Distance d from the radiation source is expressed in meter. Like in the robot 2 of FIGS. 2A-2D, the eight lamps are regularly distributed along an outer circumference of the housing 24 of the robot 2. The irradiance values were measured with a Pedak UV-C-254 handheld UV-C light meter at different distances d orthogonally with respect to the lamps, having a longitudinal shape. After fitting the measured irradiance values with a power function (I=P·d^{Q}), it was possible to find coefficients P and Q (P=0.51; Q=−1.39). This function allows calculating irradiance values I for different distances from the radiation source. Such a function may be implemented in a controlling computer program that may, in use, run on the controller 10, for determining the exposure pattern.

For a space portion at the distance d from the radiation source, the received amount of ultraviolet radiation may be determined by multiplying the irradiance (mW/cm²) to the time of exposure. The required exposure time of a space portion may depend on a required level of disinfection. Normally, the amount of inactivation of pathogens is directly proportional to the UV-C dose (which may be measured in J/m²), which is in turn a result of the UV-C intensity (also referred to as irradiance, which may be measured in W/m²) multiplied by the exposure time. As illustrated in FIG. 3, the intensity of the radiation, such as the irradiance, may be estimated to decrease as a function of distance from the position of the radiation source.

The controller may be arranged for storing disinfection information. The disinfection information may e.g. include a minimum exposure threshold (or, in other words, a minimum UV dose). Optionally, different minimum exposure thresholds are applied for different parts of the space. Thus, the minimum exposure threshold applied may vary between different space portions, or may be substantially uniform for the plurality of space portions, e.g. throughout the space. A minimum exposure threshold for a space portion associated with a surface property, may be based on the surface property. For example, for a space portion associated with a surface that has a relatively porous structure and/or with a surface having a relatively strong shadow effect, the minimum exposure threshold may be relatively high.

The disinfection information may further include a type of pathogen and/or allergen against which the space is to be disinfected, a required disinfection level, and a radiant power of the radiation source. The controller may be arranged for basing a duration of disinfecting and/or velocity of the radiation source, on the minimum exposure threshold needed for inactivating the pathogen type up to the required disinfection level. The minimum exposure threshold may be based on measurements carried out before disinfection, optionally published in literature.

The UV dose required to reach a certain level of disinfection, i.e. to inactivate a certain percentage of a particular microorganism, can be expressed as logarithmic reduction factor R. For example, R may equal 1 (90.00% inactivation), R may equal 2 (99.00% inactivation), R may equal 3 (99.90% inactivation), or R may equal 4 (99.99% inactivation). The dose required to achieve a reduction factor R=3 (99.9% inactivation) for the most studied infections-related bacteria (methicillin resistant Staphylococcus aureus, vancomycin-resistant enterococcus, Clostridium difficilis and Escherichia coli) lies between 7 and 10 mJ/cm², while the reported dose required for R=4 is between 10 and 15 mJ/cm² (see e.g. Malayeri et al., IUVA news 2016, Fluence (UV Dose) Required to Achieve Incremental Log Inactivation of Bacteria, Protozoa, Viruses and Algae; Lindblad et al., Ultraviolet-C decontamination of a hospital room: Amount of UV light needed, Burns 46 (2020), 842-849; and Spencer et al., A model for choosing an automated ultraviolet-C disinfection system and building a case for the C-suite: Two case reports, American Journal of Infection Control 45 (2017), 288-292). Other work showed that a dose of 12 mJ/cm² is enough to achieve an R=3 for vegetative bacteria, while 22-36 mJ/cm² are required to also inactivate the spores (see e.g. Nerandzic et al., Evaluation of an automated ultraviolet radiation device for decontamination of Clostridium difficile and other healthcare-associated pathogens in hospital rooms, BMC Infectious Diseases 10 (2010), 197). Several viruses, including aerosol-carried respiratory viral pathogens such as SARS and avian influenza viruses, are susceptible to UV-C doses between 1.5 mJ/cm² and 17 mJ/cm² (R=3) (see e.g. McDevitt et al., Aerosol Susceptibility of Influenza Virus to UV-C Light, Applied and Environmental Microbiology 78 (2012) 1666-1669 .

UV-C light is capable to inactivate at least two coronaviruses (SARS-COV-1 and MERS-COV) that are near-relatives of the COVID-19 virus (see e.g. Tsunetsugu-Yokota, Large-Scale Preparation of UV-Inactivated SARS Coronavirus Virions for Vaccine Antigen, SARS-and Other Coronaviruses. Methods in Molecular Biology (Methods and Protocols), vol 454. Humana Press, Totowa, NJ, 2008; and Bedell et al., Efficacy of an Automated Multiple Emitter Whole-Room Ultraviolet-C Disinfection System Against Coronaviruses MHV and MERS-COV, Infect Control Hosp Epidemiol 37 (2016), 598-599. According to the International Ultraviolet Association (IUVA), taking into account current data and empirical evidence, the average UV dose for R=1 inactivation of the virus SARS-COV-2 (causing COVID-19) is 6.7 mJ/cm² (see 2020 COVID-19 Coronavirus Ultraviolet Susceptibility; PurpleSun Technical Report; DOI: 10.13140/RG.2.2.22803.22566). Although there are no specific data available for SARS-COV-2, the required UV dose for R=3 inactivation for a wide range of tested viruses lies between 20 mJ/cm² and 80 mJ/cm² (see e.g. Nossik et al., Resistance of DNA and RNA viruses to UV radiation, proceedings of 12th World Congress on Virology, Oct. 16-17, 2017 Baltimore, USA). A dose of 50 mJ/cm² was shown to be enough for a reduction factor R=3 for SARS-COV-1, which is closely related to SARSCOV-2 (see e.g. Eickmann et al., Inactivation of three emerging viruses-severe acute respiratory syndrome coronavirus, Crimean-Congo haemorrhagic fever virus and Nipah virus-in platelet concentrates by ultraviolet C light and in plasma by methylene blue plus visible light, Vox Sanguinis 115 (2020), 146-151).

The controller 10 is further arranged for determining a target position to extend the path in the indoor space. The target position may be based on the sensor signal and on the cumulative exposure pattern in the indoor space. Determining the target position based on the sensor signal and the cumulative exposure pattern, enables the controller to set a target position for the radiation source where an improvement of the cumulative exposure pattern can be reached, and preferably can be optimized. Instead of traversing the space in a predetermined or random way, determining the target position based on the sensor signal and the cumulative exposure pattern, enables a flexible and robust manner to disinfect the space to a sufficient extent. The cumulative exposure pattern indicates which portions of the space were exposed to the ultraviolet radiation and indicates the total amount of ultraviolet radiation that was received as a result of the disinfection.

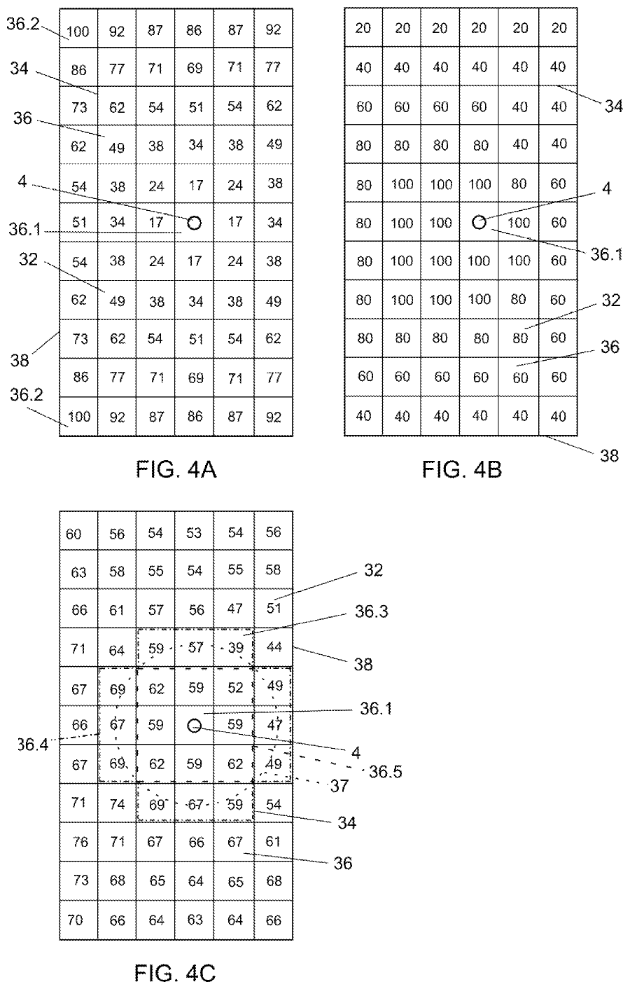

FIGS. 4A-4C illustrate an example of determining a target position for the radiation source, based on a position of the radiation source and on a cumulative exposure pattern. Such determination may be carried out by the controller 10 of a robot 2 according to the embodiment of FIG. 1 or the embodiment of FIGS. 2A-2D. FIGS. 4A-4C schematically show a space 32. A two-dimensional grid 34 may be defined in the space 32. The grid 34 defines a plurality of space portions 36, in this example rectangular elements of the grid 34. More in general, space portions may have a maximum dimension of 1 meter, preferably 0.7 meter, more preferably 0.5 meter. As an example, the element of the grid 34 may be approximately 1 meter by approximately 1 meter, or approximately 0.5 meter by approximately 0.5 meter. Alternatively, space portions may have a maximum dimension of at most 0.5 meter or at most 0.1 meter. The space portions in the space may generally have a similar size and/or shape throughout the space, or at least throughout a majority of the space.

The exposure pattern may include a plurality of space portions 36 that are associated with a position of the space portion in the space 32 and with an amount of ultraviolet radiation received in the space portion 36 as a result of the disinfecting. Thus, a position of the space portion in the space 32 and an amount of ultraviolet radiation received in the space portion 36 as a result of the disinfecting, may be associated with each of the plurality of space portions 36. Such association may be generally established by means of a data structure in a memory of the controller, wherein the plurality of space portions are individually linked to a position in the space and to the received amount of ultraviolet radiation in the space portion. The position of a space portion may be in a centre of the space portion. FIGS. 4A-4C show a space portion 36.1 wherein the radiation source is currently located. Thus, the position of space portion 36.1 may substantially coincide with a position of the radiation source. A centre of the radiation source may be closer to a centre of the space portion 36.1 than to the centre of the other space portions. The position, in particular a centre, of the space portion 36.1 may overlap with a centre of the radiation source.

FIG. 4A shows, for all other space portions, a distance to the space portion 36.1 at a certain moment in time. The distance between space portions may be measured between the centres of space portions. The distance is normalised with a maximum distance from the radiation source to a boundary 38 of the space 32, e.g. to a wall of a room. The normalised distance from a position associated with a space portion to a position 36.1 of the radiation source, is expressed in FIG. 4A on a scale that ranges from o to 100. Naturally, another scale can also be used. In the example of FIG. 4A, the space portions 36.2 have the maximum distance from the radiation source. Alternatively, another normalisation of the distance from the radiation source to a space portion can be used, such as normalisation by a dimension of the space, such as a length of the space, a width of the space, or a maximum length of a straight path in the space along which the radiation source. Alternatively, the distance to the radiation source may be normalised by a diameter D₁ or D₂ of the radiation source (indicated in FIGS. 2C and 2D).

FIG. 4B shows an example of a cumulative exposure pattern. The cumulative exposure pattern includes a plurality of the space portions 36 that are associated with a position of the space portion in the space, e.g. along the grid 34, and with an amount of ultraviolet radiation received in the space portion as a result of the disinfecting. FIG. 4B shows a received amount of ultraviolet radiation as a result of the disinfection, associated with each of the plurality of space portions. The received amount of ultraviolet radiation may be estimated by the controller, e.g. based on previously measuring and/or modelling an intensity of the ultraviolet radiation around the radiation source and on an exposure time. The exposure time may depend on a duration of movement of the radiation source along the path of the radiation source in the space 32. The received amount of ultraviolet radiation may be normalised with a maximum received amount of ultraviolet radiation. The normalised received amount of ultraviolet radiation in FIG. 4B is normalised on a scale from o to 100. Naturally, another scale can also be used. Alternatively, another normalisation of the received amount of ultraviolet radiation associated with a space portion can be used as well, such as normalisation by a minimum exposure threshold.

Optionally, the controller may be arranged for determining the cumulative exposure pattern in the space further based on spatial information and/or disinfection information, e.g. a humidity in the space and/or an amount of dust in the space, The controller may for example be arranged for determining a lowered exposure to ultraviolet radiation for positions in the space and/or space portions in the space that are associated with a higher humidity and/or a higher amount of dust. After all, a higher humidity and/or a higher amount of dust could hamper ultraviolet radiation to effectively reach a pathogen and/or allergen in the space. In this option, the minimum exposure threshold could be uniform in the space.

The controller 10 is arranged for determining the target position in a target space portion of the plurality of space portions 36. The target space portion may be associated with a lower combination of distance from the radiation source position and received amount of ultraviolet radiation, compared to one or more other space portions. In an embodiment, combination may be lower in the target space portion than in another space portion in the space 32. Thus, in the target space portion, said combination is not a maximum in the space 32. Preferably, a more restrictive criterion is applied, wherein said combination is lower than another space portion 36.4 positioned in a predetermined range 37 from said target space portion. Thus, in said range, the target space portion may not have the highest value of said combination. More preferably, said combination may be lower than another space portion 36.5 positioned adjacent to the target space portion. Generally, space portions adjacent to the target space portion may e.g. surround and/or enclose the target space portion.

Space portions adjacent to the target space portions may be neighbouring to the target space portion.

In another embodiment, the controller may be arranged for determining the target position in a target space portion 36.3 of the plurality of space portions that is associated with a lower combination of distance from the radiation source position and received amount of ultraviolet radiation, than the space portions 36.5 that are positioned adjacent to the target space portion. According to a more restrictive criterium, said combination is lower in the target space portion than in the space portions 36.4 positioned in a range from the target space portion. Said range may generally be formed or defined by a circle 37 or square, or another geometric shape, approximately centred at the position of the disinfection robot and/or radiation source. A diameter respectively side length of said circle or square, or a cross section length of said other geometric shape, may be for example 1 meter, more in general may be at least 1.5 times, or at least two times, a maximum dimension of the target grid element. Said combination may be minimal among the adjacent space portions or the space portions in the range, such as any space portion that overlaps with a circle 37 centred at the position of the target space portion. More preferably, said combination is lower than in the plurality of space portions in the space 32. Thus, in the target space portion, said combination is minimal. This latter criterion preferably is applied, in view of its efficiency.

FIG. 4C illustrates the sum of the normalised distance from the radiation source position as shown in FIG. 4A, and the normalised received amount of ultraviolet radiation shown in FIG. 4B, divided by two. Thus, FIG. 4C shows an example of a linear combination of the distance from the radiation source position and the normalised received amount of ultraviolet radiation, during disinfection. The controller may be arranged for determining the target position in a target space portion that is associated with a lower value of said sum, and hence of said sum divided by two. Preferably, the target space portion is determined so that said sum, said sum divided by two, or another linear combination, is lower in the target space portion than in a space portion positioned adjacent to said target space portion. Preferably, the target space portion 36.3 is determined so that said sum, said sum divided by two, or another linear combination, is lower in the target space portion than in all space portions positioned in an, optionally predetermined, range from said target space portion, preferably in the space 32. In the example of FIGS. 4A-4C, the target space portion is determined to be the space portion 36.3, wherein said sum is minimal in the space.

Thus, instead of being fully guided by a user of the robot, the robot 2 may generally determine the target position (or, in other words, the waypoint) autonomously. The robot 2, in particular the controller 10, may set the waypoint so that ultraviolet radiation is provided to space portions first which are relatively close to the current radiation source position and have received a relatively small amount of radiation. As a result, an amount of ultraviolet radiation that meets the exposure threshold may reach the space portions and/or surfaces in the space, at least in a part of the space.

The controller 10 may take at least two variables into account to set the next waypoint. An algorithm implemented in a computer program may, when carried out on a processor of the controller, generally look for a relatively close space portion with a relatively low amount of received ultraviolet radiation (for example expressed in ultraviolet radiation energy), and set the waypoint in that space portion. The robot may thus use two different cost maps to calculate the next waypoint. A distance cost map, an example of which is shown in FIG. 4A, which may have distance values to the radiation source normalised in a certain range, and a radiation cost map, an example of which is shown in FIG. 4B, which may have UV-C irradiance values normalised in a similar range. The new waypoint may be the minimum of an average of, or of the sum of, both cost maps, an example of which is shown in FIG. 4C.

In a variation, the controller is arranged for determining, before the radiation source has reached the target position, an updated target position. The updated target position may be based on an update of the sensor signal and/or on an update of the cumulative exposure pattern. The updated target position may be determined, more in general, in a similar way as the previous target position. The controller may be arranged for controlling, before the radiation source has reached the target position, the movement means for moving the radiation source towards the updated target position. In this way it may be prevented that the radiation source, and possibly a housing of the robot, may cover the target position. Such coverage may diminish an intensity of the ultraviolet radiation that reaches the target position.

In addition to the exposure pattern and the sensor signal, the controller 10 may be arranged for determining a target position to extend the path in the space based on other variables as well. Thus, the controller may be arranged for determining a target position to extend the path in the space further based on the disinfection information and/or spatial information with respect to the space that is to be disinfected. The spatial information may restrict the number of positions from which the target position can be selected. The controller may be arranged for receiving and/or storing such disinfection information and spatial information. The disinfection information and spatial information may include various types of information that may be taken into account by the controller, for determining the target position. The spatial information and the disinfection information may be combined. The spatial information and/or the disinfection information may be obtained by the robot autonomously, by means of the sensor means and/or by means of an external source such as a remote computer, to which the controller may be communicatively connected. Alternatively, or additionally, the spatial information and/or the disinfection information may be provided to the robot by a user of the robot.

For example, in many practical situations, there will be objects in the space, such as a table, a bed and/or a chair. As a result of such an object, the emitted ultraviolet radiation may not be completely received in some space portion. For example, an object positioned in between the radiation source and a space portion, may hinder the radiation to reach that space portion. Shadowed space portions may still receive reflected and/or diffused light (from boundaries like a wall or a ceiling, from metallic objects, etc.). Moreover, objects in the space may hinder the movement of the radiation source to, or close to, a number of space portions. Moreover, objects that are tilted (not perpendicular) with respect to incident radiation, may receive a lower irradiance. As a result, the time needed to receive a minimum required amount of ultraviolet radiation (or, in other words, a minimum required UV dose) may be increased. In order to determine, in particular estimate, the required disinfection time and/or the duration of the movement of the radiation source along the path in the space in a reliable manner, values of irradiance in tilted or shaded objects are preferably determined by experimental measures and/or may be modelled by means of a computer model, such as a ray tracing model (for an example, see Lindblad et al., Ultraviolet-C decontamination of a hospital room: Amount of UV light needed, Burns 46 (2020), 842-849). The results of such experiments and/or modelling may be stored in the controller as disinfection information, and used by the controller for determining the cumulative exposure pattern.

The spatial information may include information on a position of an object in the space and/or of a boundary of the space. The controller may be arranged for determining the target position to extend the path further based on the spatial information, by taking into account the object and/or the boundary. The exposure pattern may be associated with an object in the space and/or a boundary of the space. In that way, the exposure of the object and/or the boundary can be determined. At the same time, shadowing effects and/or reflection effects as a result of an object and/or the boundary may be estimated by the controller 10. Thus, taking into account the presence of an object and/or boundary may influence the cumulative exposure pattern in the space. Moreover, collision of the robot with objects in the space may be prevented.

The presence of objects and/or boundaries may be represented in the distance cost map (shown in FIG. 4A) and/or in the radiation cost map (shown in FIG. 4B). The controller may e.g. be arranged to associate an increased value of the distance to the radiation source with each space portion 36 (e.g. element of grid 34) that is also associated with an object and/or boundary. As a result, the controller may be reluctant to determine the target position so that it coincides with a space portion to which an object and/or boundary is associated. In other words, at positions where an object and/or boundary is located, the costs in the distance cost map may be increased.

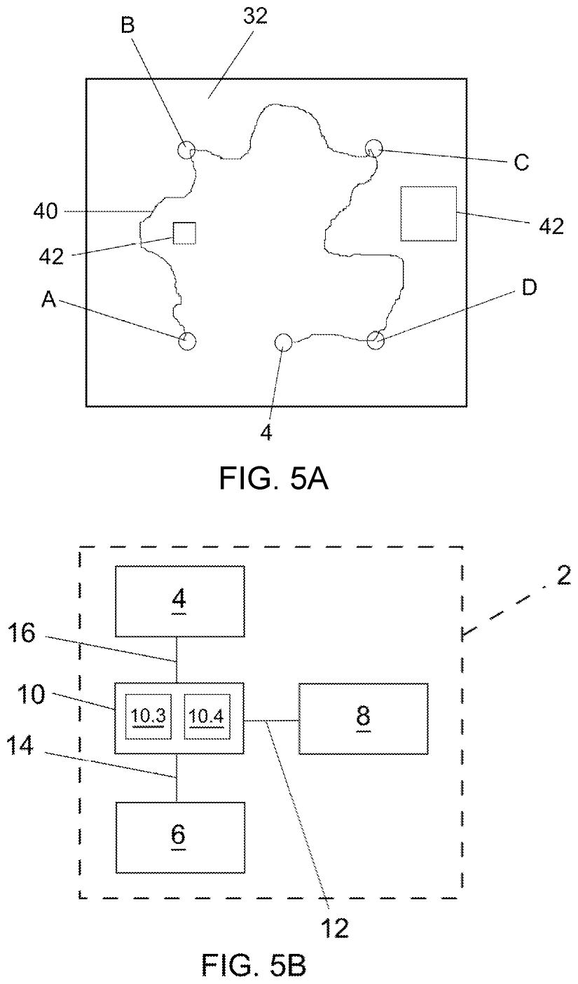

FIG. 5A shows an example of a path 40 of a radiation source in the space 32. FIG. 5B schematically illustrates a robot 2 in an embodiment of the invention including the radiation source 4, that is arranged for taking into account the spatial information for extending the path 40. The spatial information may include a plurality of positions A, B, C, D in the space that are to be passed by the radiation source 4. The position A may be regarded as a starting position of the radiation source, for disinfecting the space. The controller 10 of the robot 2 may be arranged for controlling the movement means for moving the radiation source along the path 40.

As illustrated in FIG. 5B, the controller 10 may include a global planner 10.3 for taking into account the spatial information. The global planner may be arranged for influencing the determination of the target position by a local planner 10.4, based on the spatial information. The global planner may for example be arranged to subsequently decrease the costs in the distance cost map 4B in a vicinity of the positions A, B, C, D that are to be passed by the radiation source. For example, once the radiation source has passed point B, the distance cost map 4B in a vicinity of point C is decreased and a decrease in a vicinity of point B is removed. Once the radiation source has passed the point C, the distance cost map 4B in a vicinity of point D is decreased and a decrease in a vicinity of point C is removed.

The controller 10 may include the local planner 10.4 for determining the target position (or, in other words, the waypoint). In use, the local planner 10.4 may receive, via communicative connection 12, the sensor signal that is indicative for a position of the radiation source in the space 32. The local planner 10.4 may determine the path 40 for a part of the path whose start and end are substantially determined by the global planner, in FIG. 5A e.g. between points A and B or between points B and C. The local planner 10.4 may take into account the presence of objects 42 in the space 32, such as a chair or a closet. In use, the local planner may be arranged to receive the sensor signal that is indicative for a position of the radiation source in the space 32. When a new object appears to the local planner, in use the local planner may extend the path around the object 42.

The spatial information may be provided to the robot before disinfecting starts. The spatial information that may be used by the global planner 10.3, may remain substantially unaltered during disinfecting the space 32. This may be justified for example in situations wherein the space remains according to its assumed configuration (e.g. similar to a previous disinfection of that same space). Alternatively, the controller, in particular the global planner, may be arranged to update the spatial information during disinfecting. For example, a map of the space may be updated in real time based on the sensor signal.

Thus, the global planner 10.3 and/or the local planner 10.4 may be provided with information on a position of an object in the space and/or a boundary of the space. The local planner 10.4 may be arranged e.g. for determining the target position to extend the path in the space further based on the position of the object in the space and/or the boundary of the space. Also, the global planner 10.4 may be arranged for influencing the determination of the target position to extend the path in the space further based on the position of the object in the space and/or the boundary of the space.

It may be clear from the above that, in an embodiment, the global planner may be provided with the map of the space. The global planner may be provided with the map before starting disinfection. The map may be updated, based on the sensor signal, by the controller during disinfecting. The updated map may e.g. be used by the global planner for determining the path in the space during subsequent disinfection of the space.

In a variation, the controller 10 may be arranged for estimating spatial information and/or disinfection information in the space before starting disinfecting the space. Estimating may be based on spatial information and/or disinfection information obtained during prior disinfecting. For example, an amount of pathogen in the space may be estimated based on prior disinfection runs in the space and/or in similar other spaces. Estimating may e.g. be based on one or more maps of the space obtained during prior disinfecting. For example, a presence of objects in the space and/or boundaries of the space may be estimated based on sensor signals obtained during prior disinfection runs in the space and/or in similar other spaces. Estimating may be carried out by the controller 10, in particular the global planner 10.4, and/or by an external source such as a server computer. In both ways, the global planner may be provided with a map that is estimated prior to disinfecting. Various ways of estimating that may be applied are known as such to the skilled person, such as averaging, extrapolation, such as linear extrapolation, or machine learning. Estimating may also use a priori knowledge such as functional relations between temperature and/or humidity on the one hand, and growth of pathogens and/or allergens on the other hand. Using a map that is estimated prior to disinfecting may enable improved disinfecting, in particular may improve the global planner 10.4.

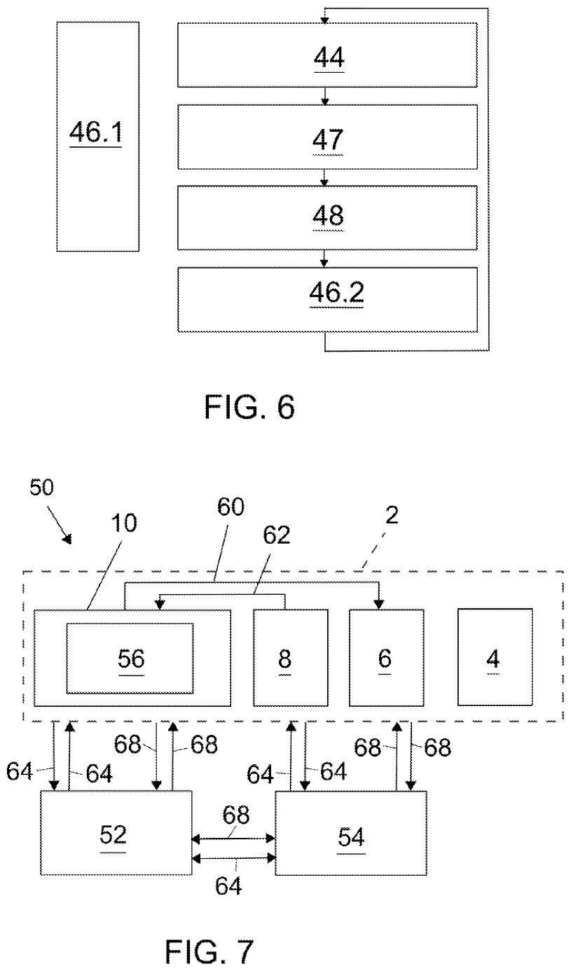

FIG. 6 illustrates a method of disinfecting, in an embodiment of the invention. The method includes disinfecting a, preferably indoor, space such as a room of a hospital or a nursing centre, by means of ultraviolet radiation. The ultraviolet radiation is emitted in the space by means of a radiation source. The radiation source is included by a disinfection robot for disinfecting the space. The robot may for example be a robot in an embodiment as described with reference to FIGS. 1-5B. However, the robot may be embodied in another way as well. Carrying out the method may be controlled, at least partly, by means of a controller of the robot. The controller may include a processor and a memory, and be arranged for running a computer program for carrying out one or more method steps (i.e., one or more parts of the method).

The method may include steps 44 of generating, by means of sensor means that are included by the robot, a sensor signal that is indicative for a position of the radiation source in the space, and receiving, by means of a controller that is included by the robot and is communicatively connected to the sensor means, the sensor signal. Steps 44 may be carried out repeatedly, yielding a sequence of positions of the radiation source.

The method may further include a step 46.1 of moving the radiation source in the space by means of movement means that are included by the disinfection robot, and controlling, by means of the controller, said moving along a path in the space. The path may be formed by a sequence of the positions of the radiation source. The step 46.1 of moving the radiation source may include intervals wherein the radiation source is static, i.e. does not change position. Such intervals in between periods wherein the radiation source does change position, are considered to be part of the moving of the radiation source. While carrying out the step 46.1 of moving the radiation source, steps 44 may be carried out repeatedly.

The method may further include a step 47 of determining, by means of the controller, a cumulative exposure pattern of the emitted ultraviolet radiation in the space. Determining the exposure pattern is based on the path, on a duration and/or velocity of movement of the radiation source along the path, and on an intensity and/or direction of the ultraviolet radiation emitted during movement along the path. Determining the exposure pattern based on the intensity of the ultraviolet radiation may take into account a decrease of the intensity with increasing distance from the radiation source, as for example shown in FIG. 3. Determining the exposure pattern based on the intensity of the ultraviolet radiation may further take into account a decreasing radiant power of the radiation source with time. Such variations in intensity are preferably determined experimentally and/or by modelling, before using the radiation source. Step 47 of determining the cumulative exposure pattern may be carried out repeatedly during the step 46.1 of moving the radiation source.

The method may further include a step 48 of determining, by means of the controller, a target position to extend the path in the space based on the sensor signal and on the cumulative exposure pattern in the space. Determining the target position may be carried out for example as described with reference to FIGS. 4A-4C. The method may further include the step 46.2 of controlling the movement means for moving the radiation source towards the target position. At a certain moment in time, moving the radiation source towards the target position to extend the path, may be considered to be carried out after the step 46.1 of moving the radiation source along the path. At a later moment in time, the previous moving of the radiation source may be considered to be part of step 46.1. Thus, the moving of step 46.1 may, partly or completely, be carried out by previous repeated movement steps 46.2 towards a previous target position.

As described with reference to FIGS. 4A-4C, the cumulative exposure pattern may include a plurality of space portions that are associated with a position of the space portion in the space and with an amount of ultraviolet radiation received in the space portion. The controller, in particular the processor of the controller, may be provided with a computer program that includes instructions that, when carried out by the processor of a controller, carry out the step of determining the target position to extend the path in the space based on the sensor signal and on the cumulative exposure pattern in the space.

In an embodiment, the method includes determining, by means of the controller, the target position in a target space portion of the plurality of space portions that is associated with a lower combination of distance from the radiation source position and received amount of ultraviolet radiation, than another space portion in the space. A more restrictive criterium for determining the target space portion may be applied, by selecting a space portion wherein said combination is lower than another space portion positioned in a range from said target space portion. Alternatively, the combination may be lower than another space portion positioned adjacent to the target space portion, e.g. is neighbouring to the target space portion. Thus, among a plurality of space portions, the target space portion may not have the highest value of said combination.

In a preferred embodiment, the target position is determined in a target space portion of the plurality of space portions that is associated with a lower combination of distance from the radiation source position and received amount of ultraviolet radiation, than a majority, preferably all, of the space portions adjacent to the target space portion. In this embodiment, a more restrictive criterium may be that said combination in the target space portion is lower than a majority, preferably all, of the space portions positioned in a range from the target space portion. It is preferred that, in the target space portion, said combination is lower than a majority, preferably all, of the plurality of space portions in the space.

FIG. 7 shows a system 50 that includes a robot 2, in an embodiment of the invention. The robot may for example be a robot in an embodiment described with reference to FIGS. 1-5B. The robot may be embodied in another way as well. The system 50 may further include a remote computer. The system 50 may include two remote computers, for example a server computer 52 and a mobile device 54. The mobile device 54 may be a mobile phone, a laptop, a tablet computer, or another mobile computing device. By means of the mobile device 54 the robot 2 may be operated from a safe distance to the radiation source 4 of the robot 2. The server computer 52 and the remote mobile device 54 may be communicatively connected to the robot for receiving information from the robot and/or for sending information to the robot. Optionally, the remote computers may be communicatively connected to each other via a communicative connection.

FIG. 7 further illustrates a flow of information that may occur in the system 50. The controller 10 may be communicatively connected to the movement means 6, for sending to the movement means a movement signal 60. Via the movement signal 60, the movement means of the robot 2 may be controlled. The movement means 6 may be controlled to control a velocity and direction of the radiation source that moves in the space. More in general, the controller may be arranged for controlling, via the movement signal 60, the movement means for moving the radiation source along a path in the space.

The controller 10 may further be communicatively connected to the sensor means for receiving a sensor signal 62 that is indicative for a position of the radiation source 4 in the space. The sensor signal may further be indicative for a position of an object in the space and/or a boundary of the space. Thus, more in general, the sensor signal may include a position signal that is indicative for a position of the radiation source 4 in the space, and may optionally include a distance signal that is indicative for a position of the object in the space and/or a boundary of the space.

The system 50 may further include a mapping module 56. The mapping module 56 may be part of the controller 10. The mapping module may be included in the housing of the robot. Alternatively, the mapping module may be remote from the housing. For example, the mapping module may be formed by a computer that is stationary while the radiation source of the robot moves through the space. The controller 10, in particular the mapping module 56 of the controller 10, may be arranged for generating and/or updating a map of the space, based on the sensor signal 62. By combining the position signal and the distance signal, a position of an object in the space and/or a boundary of the space may be determined. The map may be generated and/or updated before and/or during disinfecting the space.

In use of the system 50, spatial information 64 may be sent by the robot 2, in particular by the controller 10, more in particular by the mapping module 56, to the server computer 52. In use, the spatial information 64 may be stored on the server computer 52. The spatial information 64 may also be sent from the server computer to the robot, in particular to the controller 10 of the robot 2. For example, the spatial information may be sent to the server computer 52 during a previous disinfection cycle, and may be reloaded into the robot for use during a next disinfection cycle.

Thus, more in general, the server computer 52 and the robot 2 may be arranged for exchanging with each other the spatial information 64, or at least a part thereof. The server computer 52 may also be arranged for storing disinfection information 68. As described herein, the disinfection information may include various types of information that may be taken into account for disinfecting. For example, the disinfection information may include information on a pathogen type and/or allergen type against which the space is to be disinfected and/or information on a minimum exposure threshold. The minimum exposure threshold is the minimum amount of radiation that is to be received in order to inactivate a required percentage of the pathogen type and/or allergen type (i.e., the required disinfection level) against which the space is to be disinfected. The disinfection information may further include a minimum part of the space that is to reach the minimum exposure threshold. The minimum part of the space may for example be a part of the space where a patient normally is to be treated. Optionally, different minimum exposure thresholds are applied for different parts of the space. The disinfection information may further include information on an intensity of the radiation source, e.g. at a specified distance of the radiation source, and/or information on a radiation time of the radiation source, e.g. on a burning time of the lamps included by the radiation source. Such information may be used by the controller for determining the cumulative exposure pattern.

The minimum part of the space may optionally be determined by an intensity of use within the space. Such an intensity of use may e.g. be determined by an occupation rate, or frequency of occupation, of portions of the space by one or more persons in the space. The intensity of use may be determined by means of the sensor means, in particular by means of a camera included by the sensor means. The sensor means may include one or more cameras for determining the intensity of use in the space, prior to disinfecting. Such cameras may e.g. monitor the space during a period of time, e.g. one or more days, in order to generate a sensor signal that is indicative for the intensity of use in the space. The controller may be arranged for determining the minimum part of the space by comparing the intensity of use inferred from the one or more cameras, with at least a predetermined minimum use threshold. In the minimum part of the space, the intensity of use may be above the minimum use threshold. Outside the minimum part of the space, the intensity of use may be below the minimum use threshold. The minimum use threshold of a space portion may for example be a minimum number of persons that pass through a space portion during a day, The minimum use threshold may for example be four persons per day.

The disinfection information may be sent from the robot 2 to the server computer as well. The disinfection information may for example be sent to the server computer to verify whether the disinfection information used by the robot, is still up to date. The disinfection information sent to the server computer may also include disinfection information received by the robot from another remote computer, such as the mobile device 54. In this way, the relevant disinfection information on both remote computers can be kept aligned. More in general, both remote computers may exchange, i.e. send and/or receive, disinfection information, directly and/or via a communicative connection via the robot 2.

Like the server computer, the mobile computer 54 and the controller 10 may be arranged for exchanging with each other the spatial information 64 and/or the disinfection information 68, or at least a part thereof. In an embodiment, different types of information are exchanged via both types of remote computers. Information that remains unchanged after a plurality of disinfections may be exchanged between the server computer and the controller. Such information can be used for a plurality of spaces and for a plurality of disinfections. Preferably, the server computer 52 is communicatively connected to a plurality of robots 2. For these robots, the same disinfection information may be used, such as a minimum exposure threshold for a specific type of pathogen or allergen. Information that is specific for a disinfection, may be exchanged between the mobile device 54 and the robot 2.

A practical embodiment of use of the robot 2, may start with running on a remote computer, such as the mobile device 54, a software application and establishing a wireless communicative connection with the robot 2 and in particular with the controller 10, via the communication device 30 of the robot 2. The robot may be placed in the space. Via the remote computer, the user may instruct the robot to start mapping the space, i.e. to create a map of the space. The space map may be stored on a further remote computer, such as a server computer 52.

After mapping the space, the user may instruct the robot to start disinfecting the space. After receiving the instruction, the robot may receive the space map from the server computer. Via the remote computer operated by the user, the robot, in particular the radiation source, may be moved to a starting position. The user may further instruct the robot to start disinfecting by radiating ultraviolet radiation and moving the radiation source towards the first target position. After the robot has completed disinfection, the robot may generate a disinfection report that contains a result of the disinfection.

Surfaces in the space to be disinfected are preferably free from other loose materials like cloth, paper or ordinary glass. Such materials may block ultraviolet radiation to disinfect the surface. The robot may be used as a supplement to conventional disinfection and/or cleaning. The robot may be arranged to generate a disinfection report that contains, e.g. indicates, a result of the disinfecting. The report may, for example, include a temperature in the space, a humidity in the space, an amount of dust in the space, and/or amount of ozone in the space, before, during, and/or after disinfecting. A human may further disinfect the space, based on the report generated by the robot.

Optionally, the controller 10 may be generally arranged for controlling the movement means 6 by not starting disinfecting if the humidity is larger than an upper humidity threshold, if the amount of dust is larger than an upper dust threshold, if the amount of ozone is larger than an upper ozone threshold, and/or if the temperature is below a lower temperature threshold, and preferably is arranged in such cases for recommending drying, cleaning, ventilating, and/or heating the space, respectively. The robot 2 may be arranged to generate a disinfection report that contains such a recommendation. Thus, by determining in the space the humidity, temperature, and/or amount of dust, the disinfecting may be improved by adjusting the velocity and/or duration of the movement of the radiation source 4 along the path 40.

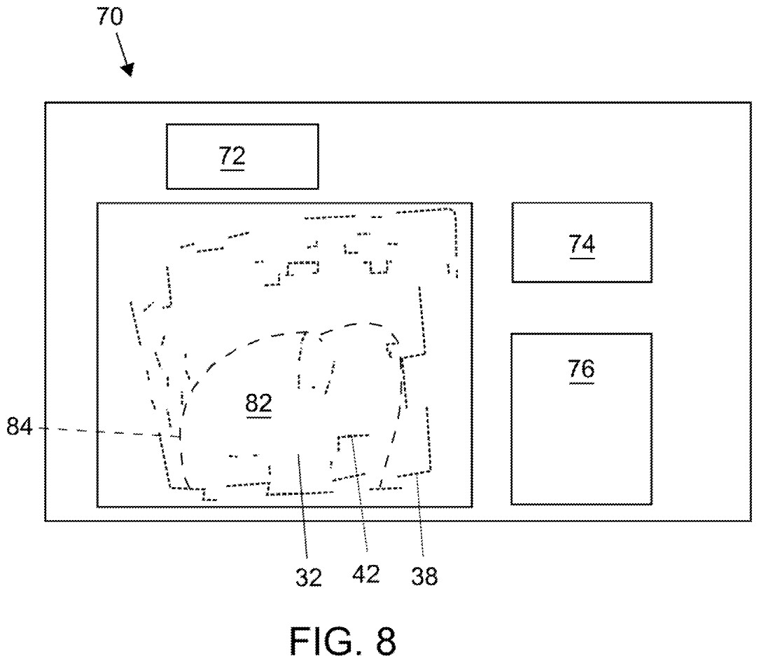

FIG. 8 shows an example of a disinfection report 70. The disinfection report may contain a section 72 that contains general data on the disinfecting, such as a date and time of disinfecting, a total duration of the disinfecting, a location of the disinfected space, and/or a type of the disinfection space. The space can for example be an office space, a medical space, or a production space. The disinfection report 70 may further contain a map 80 of the space 32. The map may e.g. include positions of objects 42 in the space and boundaries 38 of the space 32. Additionally, or alternatively, the map may include a temperature in the space, a humidity in the space, an intensity of use in the space, and/or an amount of dust in the space. The map may associate such variables to a position in the space and/or to a space portion in the space. In this way, the map may generally be arranged for representing a spatial distribution of such variables in the space. The map may further include a position of a plurality of space portions such as grid elements (not drawn in FIG. 8). By means of the map, space portions may be associated with its position and the presence or absence of an object or boundary. The example of FIG. 8 shows a map 80 that is a schematic and regularised representation obtained during an experiment carried out with a robot 2 in an embodiment of the invention.

The disinfection report further may contain a section 74 that contains an indication that relates to a part 82 of the space 32 that has reached a minimum exposure threshold, i.e. may be considered to be disinfected. The section 74 may for example contain a label that also is contained by a map 80 of the space, so that a user can recognise on the map the part of the space that was disinfected. The label may for example be a boundary 84 of the disinfected part. The disinfection report may also contain a section 76 that contains information on a type of pathogen and/or allergen against which the space is disinfected, optionally in combination with the level of disinfection that was reached. The section 76 may for example contain information such as “Covid-19 99.99%”, “Ebola 96.3%”, “Measles 94.7%”, etc. Optionally, the disinfection report may indicate parts of the space wherein the minimum exposure threshold was not reached, This can e.g. be caused by a surface property in such parts that has a relatively high minimum exposure threshold. More in general, the types of pathogen and/or allergen referred to herein may relate e.g. to Covid-19, Ebola, Measles, Influenza, Noroviruses, C-diff bacteria, Extended Spectrum Beta-Lactamase (ESBL) producing bacteria, and/or MRSA bacteria.

Thus, with reference to FIGS. 1-8, a robot for disinfection and a method of disinfection are described, that enable a path in a space wherein the ultraviolet radiation may be distributed relatively effectively and disinfection can be completed within a reasonable or relatively short time. The path may be determined taking into account shadows and reflections of one or more objects that could be present in the space, so that such shadowed portions of the space may be adequately disinfected. The robot may efficiently disinfect spaces whose configuration changes, for example in between two disinfections. One or more objects that may be present in the space do not have a predetermined and/or fixed position in the space.

Optionally, in a robot, system, or method disclosed herein, the cumulative exposure pattern may be limited, or use of the cumulative exposure pattern for determining the target position may be limited, to a part of the cumulative exposure pattern associated with a part of the space, preferably a part of the space wherein the intensity of use is above the minimum use threshold. Disinfection of the part of the space may be achieved in a smaller amount of time than disinfecting the whole space. Parts of the space where the intensity of use is relatively low may be less vulnerable for contamination and/or may be less vulnerable for infection to occur. Optionally, the controller 10 may generally be arranged for applying a mask to the cumulative exposure pattern, to the space, to the plurality of space portions, and/or to the data structure. As a result of the mask, the parts of the cumulative exposure pattern, the space, the plurality of space portions, and/or the data structure where the intensity of use is below the minimum use threshold, may be masked so that they are not taken into account by the controller 10 for determining the target position and/or and for controlling the movement means for moving the radiation source, e.g. towards the target position.

The embodiments described herein illustrate various features. Features disclosed in relation to one or more of the embodiments described herein, may be applied in other embodiments as well. The invention is not limited to the embodiments described with reference to the figures, and may be embodied in other ways as well. For example, the radiation source may be moveable relative to the housing. Movement of the radiation source relative to the housing may be controlled by the controller. SI-units are used herein, like meter (m), second(s), kilogram (kg), Joule (J), and watt (W). Length may e.g. be expressed in meter, centimetre (cm) or nanometre (nm). Time may e.g. be expressed in seconds. Energy may e.g. be expressed in millijoule (mJ). Power may e.g. be expressed in watt (W) or milliwatt (mW). Features described in relation to a robot may be applied in relation to a method or use, and vice versa. The invention is not limited to an aspect, embodiment, feature, variation, or example of the present disclosure. All kinematic inversions are considered to be inherently disclosed and to be within the scope of the present disclosure. The use of expressions like “preferably”, “more preferably”, “in particular”, “in a variation”, “e.g.”, “for example”, “such as”, “may”, “can”, “could”, “embodiment”, “aspect” etc. is not intended to limit the invention.

Use herein of terms like “a”, “an”, “the” etc. does not exclude a plurality. The term “step” may refer to any part of a method. Basing a first variable, signal, or parameter on a second variable, signal, or parameter, does not exclude that the first variable, signal, or parameter is further based on one or more other variables, signals, or parameters. A first variable, signal, or parameter may also be considered to be based an another variable, signal, or parameter, if this other variable, signal, or parameter is first converted into one or more intermediate variables, signals, or parameters. Between generating a signal and receiving the signal, the information and/or form of a signal may change. E.g., information may be added to the signal after generation of the signal, the signal may change as a result of modulation, may attenuate, may be coded, may be amplified, may be filtered, etc. A signal that is indicative for a variable, may be indicative for one or more other variables as well. For example, the sensor signal may include signals that are indicative for different variables and are obtained from different sensor devices that may be included by the sensor means, such as a camera and a device for measuring distance. Optionally, in use, one of more of the sensor devices may move together with the radiation source. Optionally, in use, one or more of the sensor devices may not move together with the radiation source, e.g. may be stationary during movement of the radiation source. Hence, more in general, a position of a sensor device may be dependent, or may be independent, of a position of the radiation source and/or of the movement of the radiation source.

In an embodiment, the sensor means include a plurality of ultraviolet radiation detector devices that form the sensor devices. Examples of such detector devices are known as such to the skilled person. The detector devices may be located at a plurality of locations in the space. The sensor signal may include a signal that is indicative for an amount of ultraviolet radiation received by the respective detector devices located at the plurality of locations in the space. The controller may be arranged for determining the cumulative exposure pattern of the emitted ultraviolet radiation in the space, based on the signal that is indicative for an amount of ultraviolet radiation received at the locations in the space. Such determining may be in combination with, or alternative to, determining the cumulative exposure pattern of the emitted ultraviolet radiation in the space based on the path, on a duration and/or velocity of movement of the radiation source along the path, and on an intensity and/or direction of the ultraviolet radiation emitted during movement along the path.

The term “determining” used herein may be interpreted broadly. Determining a position, or an amount of ultraviolet radiation, may for example be based on an estimate, model, or indication of said position or amount of ultraviolet radiation. The term “information” as used herein may refer to physical representations of such information, such as electronic signals and information that is stored in a memory or on a computer-readable medium. Thus, the term “information” may generally refer to an information signal or stored information.

1. Disinfection robot for disinfecting a, preferably indoor, space such as a room of a hospital or a nursing centre, by means of ultraviolet radiation, wherein the disinfection robot is arranged to include, and preferably includes, a radiation source that is arranged for emitting the ultraviolet radiation, wherein the disinfection robot also includes movement means for moving the radiation source in the space and sensor means for generating a sensor signal that is indicative for a position of the radiation source in the space, wherein the robot further includes a controller that is arranged for receiving the sensor signal, preferably is communicatively connected to the sensor means for receiving the sensor signal, and that is arranged for controlling the movement means for moving the radiation source along a path in the space, said path being formed by a sequence of the positions of the radiation source in the space, wherein the controller is further arranged for determining a cumulative exposure pattern of the emitted ultraviolet radiation in the space based on the path, on a duration and/or velocity of movement of the radiation source along the path, and on an intensity and/or direction of the ultraviolet radiation emitted during movement along the path, wherein the controller is arranged for determining a target position to extend the path in the space based on the sensor signal and on the cumulative exposure pattern in the space, and for controlling the movement means for moving the radiation source towards the target position.

2. Disinfection robot according to claim 1, wherein the cumulative exposure pattern includes a plurality of space portions that are associated with a position of the space portion in the space and with an amount of ultraviolet radiation received in the space portion as a result of the disinfecting, wherein the controller is arranged for determining the target position in a target space portion of the plurality of space portions that is associated with a lower combination of distance from the radiation source position and received amount of ultraviolet radiation than a majority, preferably all, of the plurality of space portions in the space and/or of the space portions in a range from the target space portion.

3. Disinfection robot according to claim 1, wherein the cumulative exposure pattern includes a plurality of space portions that are associated with a position of the space portion in the space and with an amount of ultraviolet radiation received in the space portion as a result of the disinfecting, wherein the controller is arranged for determining the target position in a target space portion of the plurality of space portions that is associated, in a range from the target space portion and/or adjacent to the target space portion, with a minimum of a combination of distance from the radiation source position and received amount of ultraviolet radiation.

4. Disinfection robot according to claim 2, wherein the combination is a substantially linear combination, such as the sum or an average, of the, preferably normalised, distance from the radiation source position and the, preferably normalised, received amount of ultraviolet radiation.

5. Disinfection robot according to claim 1, wherein the controller is arranged for determining, before the radiation source has reached the target position, an updated target position based on an update of the sensor signal and/or on an update of the cumulative exposure pattern, and for controlling, before the radiation source has reached the target position, the movement means for moving the radiation source towards the updated target position.

6. Disinfection robot according to claim 1, wherein the controller is provided with spatial information that relates to a configuration of the space, wherein the controller is arranged for determining the target position to extend the path in the space further based on the spatial information.

7. Disinfection robot according to claim 6, wherein the spatial information includes a position of an object in the space and/or a boundary of the space, wherein the controller is arranged for determining the target position to extend the path in the space further based on the spatial information by preventing overlap between the position of the radiation source and/or the target position, and the position of the object in the space and/or the boundary of the space.

8. Disinfection robot according to claim 7, wherein the controller is arranged for mapping the space by determining, based on the sensor signal, a distance to the object in the space and/or the boundary of the space.

9. Disinfection robot according to claim 7, wherein the controller is arranged for associating the cumulative exposure pattern to the object in the space and/or the boundary of the space.

10. Disinfection robot according to claim 1, wherein the controller is provided with disinfection information that relates to a quality of the disinfection, wherein the controller is arranged for determining the target position to extend the path further based on the disinfection information.

11. Disinfection robot according to claim 10, wherein the disinfection information includes a minimum exposure threshold and/or a minimum part of the space that is to reach the minimum exposure threshold, wherein the controller is arranged for determining the target position to extend the path further based on the disinfection information by extending the path at least until the amount of received ultraviolet radiation of the cumulative exposure pattern is above the minimum exposure threshold in at least the minimum part of the space.

12. Disinfection robot according to claim 11, wherein in the minimum part of the space an intensity of use is above a minimum use threshold and/or outside the minimum part of the space an intensity of use is below the minimum use threshold.

13. Disinfection robot according to claim 11, wherein the cumulative exposure pattern includes a plurality of space portions that are associated with a space portion position in the space, an amount of ultraviolet radiation received in the space portion, and a surface property in the space portion, wherein the minimum exposure threshold is based on the surface property.

14. Disinfection robot according to claim 10, wherein the disinfection information includes information on a pathogen type, allergen type, and/or required level of disinfection, wherein the controller is arranged for determining the target position to extend the path further based on the disinfection information by determining the duration and/or velocity of movement of the robot along the path based on the information about the pathogen type, allergen type, and/or required level of disinfection.

15. Disinfection robot according to claim 1, wherein the sensor means are arranged for generating a sensor signal that is indicative for a type of pathogen and/or allergen in the space, for an amount of pathogen and/or allergen in the space, for a humidity in the space, for a temperature in the space, and/or for an amount of dust in the space, wherein the controller is arranged for controlling the movement means for moving the radiation source, in particular for controlling the movement means for controlling a duration and/or velocity of moving the radiation source, along the path in the space, based on the sensor signal that is indicative for a type of pathogen and/or allergen in the space, for an amount of pathogen and/or allergen in the space, for a humidity in the space, for a temperature in the space, and/or for an amount of dust in the space.

16. Disinfection robot according to claim 1 wherein the robot includes a communication device for sending information to the robot and/or receiving information from the robot.

17. System that includes a disinfection robot according to claim 16 and a remote computer, wherein the remote computer is communicatively connected to the robot for receiving information from the robot and/or sending information to the robot.

18. System according to claim 17, wherein the remote computer is communicatively connected to a plurality of robots according to claim 14.

19. Method of disinfecting a, preferably indoor, space such as a room of a hospital or a nursing centre, by means of ultraviolet radiation that is emitted in the space by means of a radiation source that is included by a disinfection robot for disinfecting the space, the method including:
generating, by means of sensor means that are included by the robot, a sensor signal that is indicative for a position of the radiation source in the space, and receiving, by means of a controller that is included by the robot and is communicatively connected to the sensor means, the sensor signal,
moving the radiation source in the space by means of movement means that are included by the disinfection robot, and controlling, by means of the controller, said moving along a path in the space, said path being formed by a sequence of the positions of the radiation source in the space,
determining, by means of the controller, a cumulative exposure pattern of the emitted ultraviolet radiation in the space based on the path, on a duration and/or velocity of movement of the radiation source along the path, and on an intensity and/or direction of the ultraviolet radiation emitted during movement along the path,
determining, by means of the controller, a target position to extend the path in the space based on the sensor signal and on the cumulative exposure pattern in the space, and controlling the movement means for moving the radiation source towards the target position.

20. Method according to claim 19, wherein the cumulative exposure pattern includes a plurality of space portions that are associated with a position of the space portion in the space and with an amount of ultraviolet radiation received in the space portion , wherein the method includes:
determining the target position in a target space portion of the plurality of space portions that is associated with a lower combination of distance from the radiation source position and received amount of ultraviolet radiation, than a majority, preferably all, of the plurality of space portions in the space and/or of the space portions in a range from the target space portion.

21. Method according to claim 19, wherein the cumulative exposure pattern includes a plurality of space portions that are associated with a position of the space portion in the space and with an amount of ultraviolet radiation received in the space portion, wherein the method includes:
determining the target position in a target space portion of the plurality of space portions that is associated, in a range from the target space portion and/or adjacent to the target space portion, with a minimum of a combination of distance from the radiation source position and received amount of ultraviolet radiation.

22. Method according to claim 20, wherein the combination is a substantially linear combination, such as the sum or an average, of the, preferably normalised, distance from the radiation source position and the, preferably normalised, received amount of ultraviolet radiation.

23. Computer program product that includes instructions that, when carried out by a processor of a controller, carry out a step of a method according to one of claims 19-22, which step includes determining, by means of the controller, the target position to extend the path in the space based on the sensor signal and on the cumulative exposure pattern in the space.

24-25. (canceled)
